(19) United States
(12) Patent Application Publication
(10) Pub. No.: US 2016/0184364 A1
(43) Pub. Date: Jun. 30, 2016

(54) MANAGEMENT OF OSTEOARTHRITIS USING POOLED ALLOGENEIC MESENCHYMAL STEM CELLS

(71) Applicant: STEMPEUTICS RESEARCH PVT. LTD., Bangalore, Karnataka (IN)

(72) Inventors: Pawan Kumar GUPTA, Bangalore (IN); Anoop Chullikana HOUSE, Bangalore (IN); Anjan Kumar DAS, Bangalore (IN); Anish Sen MAJUMDAR, Bangalore (IN); Swathi Sundar RAJ, Bangalore (IN); Sudha BALASUBRAMANIAN, Bangalore (IN); Mathiyazhagan RENGASAMY, Bangalore (IN)

(73) Assignee: STEMPEUTICS RESEARCH PVT. LTD., Bangalore, Karnataka (IN)

(21) Appl. No.: 14/911,545

(22) PCT Filed: Aug. 14, 2014
(86) PCT No.: PCT/IB2014/063926

§ 371 Date: Feb. 11, 2016

(30) Foreign Application Priority Data
Aug. 14, 2013 (IN) 3626/CHE/2013

(51) Int. Cl. A61K 35/28; A01N 1/02; A61K 47/42; A61K 47/26; A61L 27/20; A61L 27/38; A61L 27/50; A61K 47/20; A61K 47/02; A61K 9/00

(52) U.S. Cl. CPC A61K 35/28; A61K 47/02; A01N 1/0221; A61K 47/42; A61K 9/0019; A61L 27/20; A61L 27/3834; A61L 27/3852; A61L 27/50; A61K 47/20; A61K 47/26; A61K 2035/124

(57) ABSTRACT

The present disclosure relates to a composition and method for management of Osteoarthritis, including improvement in pain and cartilage regeneration of knee joint affected by osteoarthritis. The present disclosure also relates to a kit for treating Osteoarthritis and the method of assembling the same. The present disclosure relates to a pooled allogeneic mesenchymal stromal cell composition from multiple donors with diverse HLA genotypes suitable for transplantation into a diverse population without the risk of rejection. The pooled cell composition of the present disclosure shows increased potency and higher chondrogenic differentiation potential.

### TECHNICAL FIELD

The present disclosure relates to regenerative medicine in general, while, particularly it relates to composition of pooled allogeneic bone marrow derived mesenchymal stromal cells for management of Osteoarthritis.

### BACKGROUND OF THE DISCLOSURE

Osteoarthritis (OA) is the most common disease of the joints and a leading cause of chronic disability, especially in the aged population. Mild cases of OA can be treated with a combination of non-pharmacologic (for example, physiotherapy) and pharmacologic agents to reduce pain and inflammation. However, as the disease progresses, additional aggressive treatments are required and these may include the use of intra-articular steroids or hyaluronic acid administration. Although some patients experience temporary relief, the efficacy of these interventions is not uniform and there is some debate about their effectiveness. In more advanced or severe cases of OA, knee replacement is the only viable therapeutic option. Hence, the current treatment of Osteoarthritis is aimed at reducing pain, maintaining mobility, and minimizing disability. None of these approaches can actually cause significant regeneration of the damaged cartilage.

The present disclosure aims to overcome the drawbacks observed in the currently available treatments by providing a method of improving relief from pain and regenerating the cartilage in Osteoarthritis of knee joint using pooled allogeneic Mesenchymal Stromal Cells.

### STATEMENT OF THE DISCLOSURE

Accordingly, the present disclosure relates to a composition for treating Osteoarthritis comprising pooled allogeneic bone marrow derived mesenchymal stromal cells ranging from about 10 million cells to about 60 million cells, optionally along with carrier, cyropreservant or protein, or any combinations thereof; a method of treating Osteoarthritis, said method comprising acts of administering a composition comprising pooled allogeneic bone marrow derived mesenchymal stromal cells ranging from about 10 million cells to about 60 million cells, optionally along with carrier, cyropreservant or protein, or any combinations thereof, and optionally administering hyaluronic acid to subject in need thereof for treating the Osteoarthritis; a kit for treating Osteoarthritis in subject in need thereof comprising the above composition, hyaluronic acid, optionally along with an instruction manual; and a method of assembling a kit as above comprising act of combining the above composition and hyaluronic acid, optionally along with an instruction manual.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure where:

FIG. 1 depicts that pooled cells of the cell composition have higher chondrogenic differentiation ability when compared to the individual donors.

FIG. 2 depicts effect of treatment on pain response (PAM value).

FIG. 3 depicts effect of treatment on overall pain response (PAM value).

FIG. 4 depicts gross appearances of knee articular cartilage on the femoral side of MIA injected Wistar rat at about 4, about 8 and about 12 weeks.

FIG. 5 depicts histopathological findings (H&E) of knee femoral cross section at about 4, about 8 and about 12 weeks.

FIG. 6 depicts histology of Safranin O/Fast green stained knee femoral cross sections at about 4, about 8 and about 12 weeks.

FIG. 7 depicts immunofluorocence staining for presence of human anti-nuclear antibody in knee joint specimen.

FIG. 8 depicts level of rat Collagen Type II (at about week 4).

FIG. 9 depicts a flowchart of Clinical study design on about 60 patients, wherein administration of intra-articular injection is followed by about 2 ml of hyaluronic acid.

FIG. 10 depicts graphical representation of VAS improved in (A) about 25 and 50 million dose groups and (B) about 75 and 150 million dose groups at one year follow up. The X-axis represents different visits in which the evaluations of the patients for the respective parameters are done, wherein Visit 1: baseline, Visit 4: 1 month follow-up, Visit 5: 3 months follow-up, Visit 6: 6 months follow-up and Visit 7: 1 year follow-up. The Y-axis represents score of the evaluated parameter in about a 100 mm long scale, in which zero represents no pain and 100 mm represents worst pain. Different lines represent the different groups in the study, wherein 25M: 25 million cells, 50M: 50 million cells, P1: placebo corresponding to 25 and 50M groups; 75M: 75 million cells, 150M: 150 million cells, P2: placebo corresponding to 75M and 150M groups. The vertical bars represent±Standard deviation (S.D).

FIG. 11 depicts graph showing WOMAC scores in Cohort 1 (25 M, 50M and P1), wherein (A) shows WOMAC Composite scores, (B) shows WOMAC Pain scores, (C) shows WOMAC Stiffness scores and (D) shows WOMAC Physical function scores. X-axis represents time points of evaluation, wherein Visit 1: baseline, Visit 4: 1 month follow-up, Visit 5: 3 months follow-up, Visit 6: 6 months follow-up and Visit 7: 1 year follow-up. Y-axis represents mean WOMAC values. The vertical bars represent±S.D.

FIG. 12 depicts graph showing WOMAC scores in Cohort 2 (75M, 150M and P2), (A) shows WOMAC Composite scores, (B) shows WOMAC Pain scores, (C) shows WOMAC Stiffness scores and (D) shows WOMAC Physical function scores. X-axis represents time points of evaluation, wherein Visit 1: baseline, Visit 4: 1 month follow-up, Visit 5: 3 months follow-up, Visit 6: 6 months follow-up and Visit 7: 1 year follow-up. Y-axis represents mean WOMAC values. The vertical bars represent±S.D.

FIG. 13 depicts graph showing improvement in all components of ICOAP pain scores in Cohort 1 (25 M, 50M and P1), wherein (A) shows ICOAP Total scores, (B) shows ICOAP Constant pain scores and (C) shows ICOAP Intermittent pain scores. X-axis represents time points of evaluation, wherein Visit 1: baseline, Visit 4: 1 month follow-up, Visit 5: 3 months follow-up, Visit 6: 6 months follow-up and Visit 7: 1 year follow-up. Y-axis represents mean ICOAP values. The vertical bars represent±S.D.

FIG. 14 depicts graph showing no significant change in ICOAP pain scores in Cohort 2 (75M, 150M and P2), wherein (A) shows ICOAP Total, (B) shows ICOAP Constant and (C) shows ICOAP Intermittent. X-axis represents time points of evaluation, wherein Visit 1: baseline, Visit 4: 1 month follow-up, Visit 5: 3 months follow-up, Visit 6: 6 months follow-up and Visit 7: 1 year follow-up. Y-axis represents mean ICOAP values. The vertical bars represent±S.D.

### DETAILED DESCRIPTION OF THE DISCLOSURE

The present disclosure relates to a composition for treating Osteoarthritis comprising pooled allogeneic bone marrow derived mesenchymal stromal cells ranging from about 10 million cells to about 60 million cells, optionally along with carrier, cyropreservant or protein, or any combinations thereof.

The present disclosure also relates to a method of treating Osteoarthritis, said method comprising acts of administering a composition comprising pooled allogeneic bone marrow derived mesenchymal stromal cells ranging from about 10 million cells to about 60 million cells, optionally along with carrier, cyropreservant or protein, or any combinations thereof, and optionally administering hyaluronic acid to subject in need thereof for treating the Osteoarthritis. In an embodiment of the present disclosure, the mesenchymal stromal cells range from about 20 million cells to about 50 million cells, more preferably at about 25 million cells.

In another embodiment of the present disclosure, the pooled allogeneic mesenchymal stromal cells are obtained by combining mesenchymal stromal cells from at least two donors, preferably about 2 donors to about 10 donors, more preferably about three donors.

In yet another embodiment of the present disclosure, the pooled allogeneic mesenchymal stromal cells have plurality of HLA genotypes at loci selected from group comprising HLA-A, HLA-B, HLA-C, HLA-DQ and HLA-DR or any combinations thereof.

In still another embodiment of the present disclosure, the pooled mesenchymal stromal cells have sGAG (sulfated glycosaminoglycan) value ranging from about 25 μg to about 50 μg.

In still another embodiment of the present disclosure, at least 80% of the mesenchymal stromal cells are positive for cell specific markers selected from group comprising CD 73, CD 105, CD 44, CD90 and CD166 cells or any combinations thereof; and at least 90% of the mesenchymal stromal cells are negative for markers selected from group comprising CD34, CD₄₅, CD₁₃₃, CD14, CD19 and HLA-DR or any combinations thereof.

In still another embodiment of the present disclosure, the carrier is multiple electrolyte solution at a concentration ranging from about 80% to about 90%; the cyropreservant is Dimethyl Sulfoxide (DMSO) at concentration ranging from about 2% to about 10%, or cryopreservation medium comprising DMSO at concentration ranging from about 2% to about 10%; the protein is human serum albumin (HSA) at a concentration ranging from about 1% to about 6%.

In still another embodiment of the present disclosure, the composition is formulated as a formulation selected from group comprising aqueous suspension, emulsion, drop, emulsion in hard or soft gel capsule, elixir, lyophilized cell powder and cell spray or any combinations thereof, preferably aqueous suspension.

In still another embodiment of the present disclosure, the composition is administered as single dose or multiple doses, preferably as single dose, at one or multiple sites through mode selected from group comprising intraarticular administration, intramuscular administration, intravenous administration or any combinations thereof, preferably intraarticular administration.

In still another embodiment of the present disclosure, the subject is mammal selected from a group comprising human, horse, dog, camel, preferably human.

In still another embodiment of the present disclosure, the hyaluronic acid is administered to the subject prior to or post administering the composition comprising pooled mesenchymal stromal cells; preferably post administering the composition comprising pooled mesenchymal stromal cells.

In still another embodiment of the present disclosure, the hyaluronic acid is at an amount ranging from about 0.5 to about 6 ml, preferably about 2 ml; and the hyaluronic acid is administered at time duration ranging from about 30 seconds to about 24 hours, preferably about 30 seconds prior to or post administering the composition.

The present disclosure also relates to a kit for treating Osteoarthritis in subject in need thereof comprising composition as above; and hyaluronic acid; optionally along with an instruction manual.

The present disclosure also relates to a method of assembling a kit as above, said method comprising act of combining a composition as claimed in claim 1; and hyaluronic acid; optionally along with an instruction manual.

The present disclosure relates to use of pooled allogeneic mesenchymal stromal cells with diverse HLA genotyping representation for management of Osteoarthritis, including improvement in pain and cartilage regeneration of the knee joint affected by Osteoarthritis and prevention of further progress or manifestation of the condition. It further discloses the dosage and the mode of administration of the stromal cells to get the desired effect of improvement in pain and cartilage regeneration at the knee joint.

As used herein, the terms “mesenchymal stromal cell”, “mesenchymal stem cell” and MSCs are employed interchangeably within the instant disclosure.

As used herein in an embodiment, the terms “cell composition”, “Investigation Medicinal product (IMP)”, “final MSC composition”, “composition of the present disclosure” as used in this disclosure mean the final product comprising allogeneic pooled MSC from multiple donors with diverse HLA genotyping representation, optionally along with cryopresevation medium/solution, carrier, protein, pharmaceutically acceptable excipients or any combinations thereof; or the allogeneic pooled MSC from multiple donors with diverse HLA genotyping representation alone.

As used herein, the term “cryopreservation medium/solution” means a composition/solution used for preservation of cells for longer duration/shelf life. The alternate terms used are ‘freeze media’, ‘freezing mixture’, ‘pre-formulated ready to use preservation mixture’ all of which shall mean cryopreservation medium/solution. More specifically, in the present disclosure, MSCs are cryopreserved using a cryopreservation medium which comprises of components such as but not limiting to serum, carrier and cryoprotectant. In yet another embodiment of the present disclosure, the MSCs are cryopreserved using commercially available pre-formulated ready to use preservation mixture or solution. The cell composition is cryopreserved in liquid nitrogen at −196° C. until use. On requirement, the cells are thawed and used as per the patient dosage requirement decided by the clinician.

In an embodiment of the present disclosure, the term “managing” or “management” includes preventing, treating or healing of a disease condition or disorder or ill effects or side effects. The term also encompasses maintenance of the optimum state and prevention of the further progress in the disease condition or disorder or ill effects or side effects. Further, in a non-limiting embodiment, the present disclosure also envisages treating the said disorder by administering therapeutically effective/efficacy dosage of the cell composition. Further, “treating” or “treatment” refers to decreasing the risk of death due to a disease or disorder, delaying the onset of a disease or disorder, inhibiting the progression of a disease or disorder, partial or complete cure of a disease or disorder and/or adverse effect attributable to the said disease or disorder, obtaining a desired pharmacologic and/or physiologic effect (the effect may be prophylactic in terms of completely or partially preventing a disorder or disease or condition, or a symptom thereof and/or may be therapeutic in terms of a partial or complete cure for a disease or disorder and/or adverse effect attributable to the disease or disorder), relieving a disease or disorder (i.e. causing regression of the disease or disorder).

The present disclosure further relates to a method of managing Osteoarthritis in a subject having or suspected of having the Osteoarthritis, said method comprising act of administering a therapeutically effective amount of a cell composition

In an embodiment the cell composition comprises of allogeneic MSC with diverse HLA allele representation, Cryopreservation solution, or a combination of both. The cell composition is administrated using carrier.

The human MSCs are present as a rare population of cells in bone marrow, representing 0.001 to 0.01% of the nucleated cells, but they rapidly grow in culture without losing their stemness. MSCs can be expanded in vitro ≧2 million fold and retain their ability to differentiate into several mesenchymal lineages. MSCs differentiate not only into mesodermal lineage such as bone, cartilage and adipose tissue but also transdifferentiate into other lineages like neurons and endothelial cells. Adult MSC do not express human leukocyte antigen (HLA) class II antigens of the cell surface and do not elicit a proliferative response from allogeneic lymphocytes, thus proving that the cells are not inherently immunogenic. The MSCs also do not express co-stimulatory molecules CD80, CD86, CD40 or CD31 (PECAM-1), CD18 (LCAM) and CD56 (NCAM-1) and hence do not activate allo-reactive T cells. The cell composition of the present disclosure comprise allogeneic MSCs which do not induce lymphocyte proliferation when used in a mixed lymphocyte reaction (MLR), which is an in-vitro model of immune cell activation.

In an embodiment of the present disclosure, the multipotent mesenchymal stromal cells are derived from source selected from a group comprising but not limiting to bone marrow, adipose tissue, Wharton's jelly and dental pulp, or any combination thereof, preferably bone marrow. In an embodiment as described above, the mesenchymal Stromal cells of the instant disclosure are allogeneic. In an embodiment, allogeneic MSCs refer to MSCs which are derived from individual(s)/donor(s) other than the recipient, however belonging to the same species. The mesenchymal stromal cells used are derived from bone marrow of multiple healthy donors as per statutory requirement with proper informed consent and approval. In another embodiment of the present disclosure, the bone marrow derived MSCs are obtained by ex-vivo culturing or cell banks.

In an embodiment of the present disclosure, the effective number of pooled mesenchymal stromal cells in the cell composition ranges from about 10 million cells to about 60 million cells, preferably about 20 million cells to about 50 million cells, more preferably at about 25 million cells.

In an exemplary embodiment of the present disclosure, about 25 million pooled mesenchymal stromal cells are present in about 2 ml of carrier. In an exemplary embodiment, about 50 million pooled mesenchymal stromal cells are present in about 2 ml of the carrier.

In another embodiment of the present disclosure, the method of management of OA is using the cell composition of the present disclosure with Hyaluronic acid (HA). The HA administration can be either before or after administration of the cell composition.

In an embodiment of the present disclosure, the HA is used as biomaterial for tissue regeneration in combination with pooled MSCs. HA plays a role in visco supplementation. Administration of HA alone in OA subject does not result in regeneration of the affected cartilage. In the present disclosure the management of OA is by using the cell composition which helps in regeneration of cartilage. HA is administered in OA subject(s) post or pre administration of the composition of the present disclosure to act as a biomaterial/scaffold. The HA follows the cells of the composition and co-localises with the MSC at the joints and facilitate better engraftment and cartilage regeneration.

In an embodiment of the present disclosure, molecular weight of the hyaluronic acid administered to the subject prior to or post administration of the composition can be low molecular weight or high molecular weight. In an exemplary embodiment of the present disclosure, molecular weight of the hyaluronic acid can be below about 300 KDa or ranging from about 500,000 Dalton to about 730,000 Dalton.

In another embodiment the route of administration is parental route which includes intra-articular, intramuscular, intravenous and other routes known and general used by the practising doctor.

In an embodiment of the present disclosure, the subject is mammal selected from a group comprising but not limiting to human, horse, dog, camel, preferably human.

In an embodiment of the present disclosure, cartilage biomarker/indicator are selected from a group comprising but not limiting to Collagen, Aggrecan, HSPG and COMP is secreted in the knee joint of the subject administered with the composition comprising pooled BM-MSCs (Bone Marrow derived MSCs) of the present disclosure.

The cartilage biomarkers/indicators Aggrecan, HSPG and type II collagen are the most abundant proteins/proteoglycans found within the ECM in the articular cartilage and they are linked together by a number of collagen-binding proteins including cartilage oligomeric matrix protein (COMP). These matrix components are degraded or their production is hampered in subjects having OA, as the disease progresses.

Bone marrow derived MSCs have several advantages over other type of stem cells such as:

a. Non embryonic source,
b. Reduced likelihood of neoplasia,
c. Ease of isolation,
d. High expansion potential,
e Immune privileged,
f. Genetic stability,
h. Reproducible characteristics,
i. Compatibility with tissue engineering principles and
j. Potential to enhance repair in many vital tissues.

The cell composition of present disclosure show low immunogenicity and has been demonstrated in in-vivo in animal models using infusion of allogeneic MSCs. The injections of allogeneic MSCs do not stimulate the formation of allo-specific antibodies and do not lead to a T cell sensitization of the recipient to alloantigen in different animal models. Studies have also shown that the MSCs possess the ability to engraft, persist and function in an unrelated mismatched allogeneic host.

In an embodiment of the instant disclosure, the mesenchymal stromal cells (MSCs) of the cell composition are isolated from bone marrow of multiple donors preferably 3 donors and subjected to various processes to store or cryopreserve said cells. In an embodiment of the present disclosure, the pooled mesenchymal stromal cells employed for the management of Osteoarthiritis are derived from such stored or cryopreserved forms. The MSCs isolated from bone marrow of each individual donor are passaged and cultured to obtain Master Cell Bank composition (MCB). Said MCB comprising MSCs isolated and cultured from individual donors and is cyropreserved in Fetal Bovine Serum (FBS) and Dimethyl Sulphoxide (DMSO). Preferably, the MCB comprises MSCs of individual donors, cyropreserved in FBS at a concentration of about 90% and DMSO at a concentration of about 10%. The Mesenchymal Stromal Cells are pooled from multiple donors, preferably at least two donors to prepare the cell composition. Pooling can be defined as combining/mixing of MSCs of at least two donors from MCB in manufacturing process. The pooled MSC and subsequently passaged to arrive at a Working Cell Bank composition (WCB). WCB comprising of pooled MSCs, FBS and DMSO. Further, the said WCB preferably comprises pooled MSCs, cyropreserved in FBS at a concentration of about 90% and DMSO at a concentration of about 10%.

The aforesaid WCB is further subjected to passaging/culturing, and washing process to remove presence of xeno or other impurities to obtain a final therapeutic composition/cell composition/Investigational medicinal product (IMP)/Investigational product (IP)/final MSC composition comprising pooled allogeneic mesenchymal stromal cells with diverse HLA genotyping representation cryopreserved in a commercial available cyropreservation solution or cryopreservation solution comprising of Plasmalyte A, HSA and DMSO until further use. Optionally other pharmaceutically acceptable excipients/additives can be used for administration of the cell composition. In an embodiment of the present disclosure, the cell composition employed for the management of osteoarthritis is derived from such compositions.

### Characterization of Pooled MSCS of Cell Composition:

The pooled MSC's of the cell composition are characterised by HLA typing, markers and Chondrogenic (also referred to as chondro) differentiation potential of MSCs obtained from individual donors versus pooled cells.

### HLA Typing:

The HLA genotypes expressed by MSC from the individual donors contributing to the MCB are presented in Table 1.

The most common HLA genotypes in the global population are HLA-A*02, HLA-B*40 and HLA-DRB1*15 with a worldwide distribution of 20-28%, 5-20% and 10-18% respectively. HLA-DRB1*07, HLA-DRB1*11, HLA-DRB1*15 and HLA-DQB1*06 are genotypes frequently found in Asian and Oriental populations. The most frequent HLA genotypes in the Indian population are HLA-A*02, HLA-A*11, HLA-A*24; HLA-B*40, HLA-B*44; HLA-C*04 and HLA-C*07. HLA A*02-B*40-DRB1*15 genotypes are also found with high frequency in Indian and Pakistani populations. From Table 1 it can be inferred that the most frequently expressed HLA genotypes are represented in all the three donors from which the allogeneic MSCs employed in the cell composition of the present disclosure are obtained. Therefore, the pooled MSCs obtained by mixing the MSCs of these three donors represents most of the widely distributed HLA genotypes and is therefore suitable for transplantation into a diverse population without risk of rejection.

TABLE 1
HLA Typing

| | | | | HLA- | HLA- | |
|---|---|---|---|---|---|---|
| Donor | HLA-A | HLA-B | HLA-C | DRB1 | DQB1 | Others |
| Donor 1 | 11, 33 | 40, 44 | 04, 07 | 11, 07 | 02, 02 | DRB3, DRB4 |
| Donor 2 | 02, 24 | 07, 07 | 03, 07 | 15, 14 | 05, 06 | DRB5, DRB3 |
| Donor 3 | 02, 33 | 07, 35 | 07, 14 | 01, 15 | 05, 06 | DRB5 |

### Markers:

The MSC of the cell composition are characterized and the cells are fibroblastic and spindle shaped in active growing condition showing 80% viability. The MSC population in the cell composition are at least 80% positive for CD73, CD105, CD44, CD90 and CD166 and at least less than 10% of cells are negative for CD34, CD45, CD133, CD14, CD19 and HLA-DR in other words at least 90% of the cell are negative for CD34, CD45, CD133, CD14, CD19 and HLA-DR.

### Chondrogenic Differentiation Potential of Individual Versus Pooled Cells:

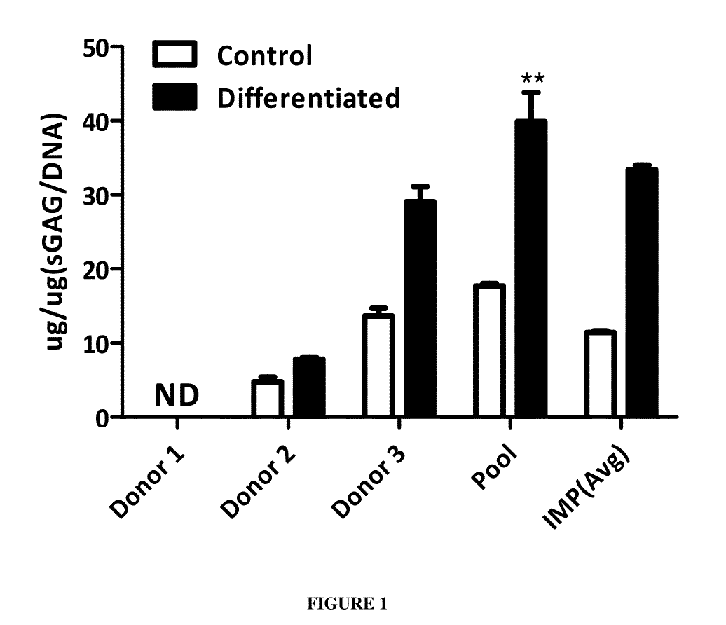

Pooled mesenchymal stromal or stromal cells (MSCs) of the cell composition are the agents of connective tissue homeostasis and repair. Osteoarthritis (OA) is a result of degeneration and failure to repair connective tissues. MSCs have been implicated in the pathogenesis of OA and, in turn, progression of the disease has been shown to be therapeutically modulated by MSCs. The potency of pooled BM MSCs of the present cell composition in OA has been established by the biochemical surrogate assay for measuring the sGAG (sulfated glycosaminoglycan) which is one of the important cartilage components and comprises of about 3-6% of total cartilage components. sGAG estimation is performed using Blyscan Assay, which is a quantitative dye-binding method. The sGAG levels for individual donors (donor 1, donor 2, and donor 3) is compared versus pooled cells of the cell composition after subjecting them to chondrogenic differentiation. Quantification clearly reveals that pooled cells of the cell composition have higher sGAG content when compared to the individual donors and one of the donors (donor1) did not have measurable sGAG level at P5 (passage 5). Thus, pooled cells have better chondrogenic differentiation potential when compared to the individual donors, FIG. 1.

### Formulation of Pooled MSC:

The formulation or composition of the present disclosure employed towards management of OA comprises allogeneic pooled MSCs optionally along with pharmaceutically acceptable excipient(s)/additive(s). In another embodiment of the present disclosure, the pharmaceutically acceptable excipient is selected from a group comprising carrier, cyropreservant, serum and pre-formulated ready to use cryopreservation mixture, or any combination thereof. In an embodiment, the carrier is Multiple Electrolytes Injection such as PlasmaLyte A, cryoprotectant is DMSO, Serum is human serum albumin (HSA) and the pre-formulated ready to use cryopreservation mixture is animal protein-free defined cryopreservation medium. In an embodiment of the present disclosure, the said composition/formulation is further diluted with carrier/vehicle (such as PlasmaLyte-A) and employed for administration purpose OR the formulation or composition comprises pooled MSCs along pre-formulated ready use commercial cyropreservation mixture selected from a group comprising CryoStor® family—a commercially available animal protein-free defined cryopreservation medium from Biolife Solutions: such as: CyroStor5 (CS5)—an optimized freeze media pre-formulated with 5% DMSO, CyroStor10 (CS10)—an optimized freeze media pre-formulated with 10% DMSO, CyroStor2 (CS2)—an optimized freeze media pre-formulated with 2% DMSO] and hypothermosol wherein said composition/formulation is further diluted with carrier/vehicle (such as PlasmaLyte-A) and employed for administration purpose. In a preferred embodiment, the cell composition is prepared by employing pooled mesenchymal stromal cells, human serum albumin, plasmaLyte-A and DMSO wherein, the said cell composition is further diluted using a carrier/vehicle such as PlasmaLyte-A to obtain the final cell composition for administration. In another preferred embodiment, pooled Mesenchymal stromal cells are cryopreserved in commercially available pre-formulated ready to use preservation mixture such as cyrostor-5 (CS5), CyroStor10 (CS10), CyroStor2 (CS2) and/or hypothermosol wherein, the said cell composition is further diluted using a carrier/vehicle for administration.

As described above, the pooled bone marrow derived Mesenchymal Stromal Cells of the present disclosure are either in combination with excipients selected from a group comprising carrier, serum and cryopreservant, optionally along with other pharmaceutically acceptable excipients/additives OR the cell composition comprises Mesenchymal Stromal Cells in a pre-formulated ready to use preservation mixture such as cyrostor-5 (CS5), CyroStor10 (CS10), CyroStor2 (CS2) and/or hypothermosol.

The cell composition is used in combination with suitable biomaterial which is administered prior to the cell composition administration or post the cell composition administration. The suitable biomaterial is selected from a group comprising Hyluronic acid or Hydrogel. In an embodiment of the present disclosure, the hydrogel is formulated from but not limiting to Poly (lactic-co-glycolic acid) (PLGA), Alginate, Chitosan, or any combinations thereof.

### Dosage of the Pooled MSC:

In another embodiment of the present disclosure, the number of allogeneic pooled Mesenchymal Stromal Cells required to manage Osteoarthritis ranges from about 10 million to about 60 million cells. In a preferred embodiment, the number of allogeneic pooled Mesenchymal Stromal Cells required to manage Osteoarthritis in the injectable suspension ranges from about 20 million to about 50 million cells. In another embodiment, the number of allogeneic pooled Mesenchymal Stromal Cells required to manage Osteoarthritis in the injectable solution/the cell composition is about 10 million cells or about 12.5 million cells or about 25 million cells about 30 million cells or about 35 million cells or about 40 million cells or about 45 million cells or about 50 million cells or about 55 million cells or about 60 million cells. In a most preferred embodiment, the number of allogeneic pooled Mesenchymal Stromal Cells required to manage Osteoarthritis is about 25 million cells.

In another embodiment, cell dosage to be injected to the patient is decided by the clinician depending on various parameters such as age, manifestation of disease condition etc.

### Route of Administration:

In another embodiment of the present disclosure, the cell composition is administered as a single dose or multiple doses at one or multiple sites through modes selected from a group comprising intra-muscular administration, intra-articular administration, intra-venous administration or through any other parenteral mode of administration known to a person skilled on the art for administration of such Mesenchymal Stromal Cells. In a preferred embodiment of the present disclosure, cell composition is administered through intra-articular administration.

In a specific embodiment of the present disclosure, the allogeneic pooled Mesenchymal Stromal Cells (MSCs) possess numerous advantages when compared to single donor derived MSCs. Pooled MSC composition of the present disclosure have significantly improved immunomodulatory characteristics thus ensuring that there is minimal biological variability/immunological reaction in the recipient post administration of pooled MSC composition.

Therefore, the various advantages of using the cell composition comprising pooled MSC along with the carriers/excipient are as follows:
a. Individual variability minimized;
b. diverse HLA allele representation;
c. Consistent non-immunogenic & immunosuppressive properties;
d. Broader Cytokine/Growth Factor (GF) array;
e. Donor specific advantage;
f. Increased potential for various disease indications; and
g. Large product doses compared to single donor.

In an en embodiment, the number of allogeneic pooled Mesenchymal Stromal Cells required in the cell composition to manage Osteoarthritis ranges from about 10 million to about 60 million cells. As an illustrative example, the present specification depicts cell composition comprising about 25 million cells and 50 million cells. However, the present disclosure is not restricted to these values but covers under its purview the range from about 10 million to about 60 million cells.

In an embodiment of the present disclosure, the experimental results provided in the present disclosure depict mean and Standard deviation (SD) values in the format of—mean (SD). For example, for the value 60.9 (19.7), 60.9 indicates the mean and (19.7) indicates the SD.

In an embodiment, it has to be understood that though considerable emphasis has been placed herein on the particular features of this disclosure, various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

Additional embodiments and features of the present disclosure will be apparent to one of ordinary skill in art based upon description provided herein. The embodiments herein and the various features and advantageous details thereof are explained with reference to the non-limiting embodiments in the description. Descriptions of well-known/conventional methods and techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the following examples should not be construed as limiting the scope of the embodiments herein.

### Example 1

### In-Vivo Study to Evaluate the Effect of Pooled Mesenchymal Stromal Cells in Cartilage Regeneration in Osteoarthritis

An in vivo study is performed to evaluate the effect of pooled mesenchymal stromal cells in cartilage regeneration in an Monosodium Iodoacetate (MIA) induced Osteoarthritis (OA) animal model.

### Method:

Single intra-articular injection of Monosodium Iodoacetate (MIA) into the femorotibial joint of rodents (monosodium iodoacetate; Sigma Cat No. I9148-5G) causes cartilage damage and degeneration in the rat knee joint. MIA—an inhibitor of glycolysis promotes loss of articular cartilage similar to that noted in human Osteoarthritis. Articular cartilage is a tissue type that is poorly supplied by blood vessels, nerves and the lymphatic system and it has a very limited capacity for repair after injury.

In an embodiment of the present disclosure, pooled Mesenchymal stromal cells are used to ameliorate the cartilage damage by its capability of regenerating cartilage cells and in management of Osteoarthritis. The endpoint parameters like pain response, cartilage damage scoring and levels of various biomarkers determine and confirm the role of pooled Mesenchymal stromal cells in the experimental model of osteoarthritis.

### Experimental Procedure:

About 80 healthy male Wistar rats are selected for the experiment. About six animals are injected with about 50 μL of normal saline intra-articularly in both knee joints and allotted to group G1 which serves as sham control. In an embodiment of the present disclosure, the normal saline contains about 0.9% Sodium Chloride Injection. Specifically, an USP of normal saline contains about 9 g/L Sodium Chloride injection with an osmolarity of about 308 mOsmol/L (calc). It contains about 154 mEq/L sodium and about 154 mEq/L chloride.

The remaining 74 animals are injected with about 1 mg of MIA prepared in about 50 μL multiple electrolyte solution viz. PlasmalyteA intra-articularly in both the knee joints. Animals are housed in individually ventilated cages and weekly observed for pain response at the knee joint using PAM (Pressure Application Meter) method.

About 3 weeks after MIA injection, animals showing pain threshold are considered as having developed Osteoarthritis and are used for grouping. Sixty animals are selected, randomized and grouped based on PAM score of the individual animals (Pain threshold—g force) obtained.

Animals are grouped into four different treatment groups (as per table 2), where group G2 animals serve as a disease control which receives only multiple electrolyte solution (PlasmaLyte A), group G3 animals serve as Hyaluronic Acid (HA) control and receive multiple electrolyte solution (PlasmaLyte A) & HA, group G4 animals are treated with high dose of preparation of the present disclosure, which comprises suspension of about 1.3×10⁶ cells of pooled Bone Marrow derived Mesenchymal Stromal Cells and HA—called BMMSC-H, group G5 animals are treated with low dose of preparation which comprises suspension of about 0.6×10⁶ cells of pooled Bone Marrow derived Mesenchymal Stromal Cells and HA—called BMMSC-L.

All the injections are given through intra-articular route (IA) in both knee joints. Group G1 serves as sham control that receives only multiple electrolyte solution (PlasmaLyte A). The volume for IA injection of multiple electrolyte solution (PlasmaLyte A)/HA/BM-MSC-H /BM-MSC-L preparation is about 30 μL in each knee joint. The concentration of HA is about 10 mg/ml and each knee joint receives about 0.30 mg of HA.

TABLE 2
Grouping and dosing of Animals

| | | Dose of | | | No. of Animals/scarification | | |
|---|---|---|---|---|---|---|---|
| Group | Treatment | BMMSC | Volume | Route | Week 4 | Week 8 | Week 12 |
| G1 | Saline + | NA | 50 μL + 60 μL | i.a. | 2 | 2 | 2 |
| | MES(PlasmaLyte | | | | | | |
| | A) | | | | | | |
| G2 | MIA + | NA | 50 μL + 60 μL | i.a. | 3 | 6 | 6 |
| | MES(PlasmaLyte | | | | | | |
| | A) | | | | | | |
| G3 | MIA + | NA | 50 μL + 30 μL + | i.a. | 3 | 6 | 6 |
| | MES(PlasmaLyte | | 30 μL | | | | |
| | A) + HA | | | | | | |
| G5 | MIA + BMMSC-L + | 0.6 × 10⁶ | 50 μL + | i.a. | 3 | 6 | 6 |
| | HA | | 30 μL + 30 μL | | | | |
| G4 | MIA + BMMSC-H + | 1.3 × 10⁶ | 50 μL + | i.a. | 3 | 6 | 6 |
| | HA | | 30 μL + 30 μL | | | | |

HA: Hyaluronic Acid
i.a.: Intra articular
MES: Multiple Electrolyte Solution,
H: High dose
L: Low dose,
NA—Not Applicable

For groups G3 to G5, about 30 μL of HA is injected at each knee joint before administration of multiple electrolyte solution (PlasmaLyte A) or pooled BM-MSC. The volume of MES (PlasmaLyte A) for group G1 and G2 is about 60 μL. Groups G2-G5 contain about 15 animals each, out of which about 3 animals are assigned for 4 week termination, about 6 animals are assigned for 8 week termination and remaining about 6 animals are assigned for 12 week termination. Sham control group contains about 6 animals wherein about two animals are assigned for each time points. The termination points are considered from the day of BM-MSC administration.

In the third week of MIA injection and on 3^{rd} day of BM-MSC administration, cyclosporine A (CsA) is injected subcutaneously for all the experimental animals at a dose of about 10 mg/kg body weight for one week, after which CsA is administered orally at a dose of about 10 mg/kg body weight till the end of the experiment. The CsA is used to increase the engraftment of human BM-MSCs in xenogeneic model.

### Example 1.1

### Pain Measurement

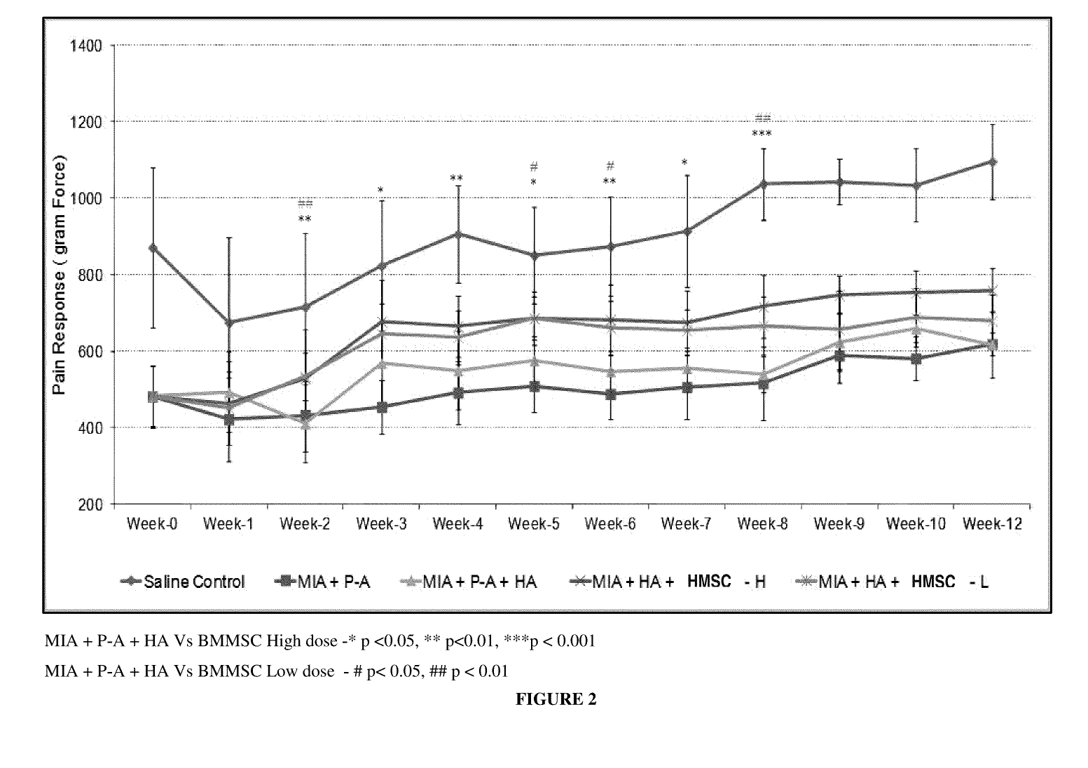

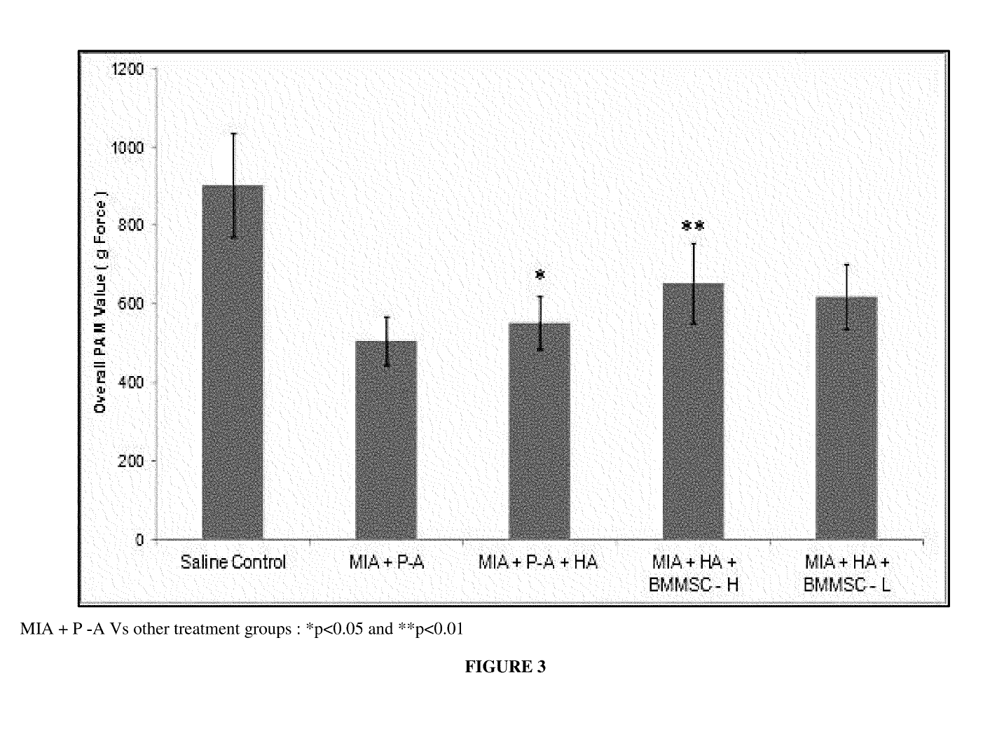

Pain in the knee joint is measured by Pressure Application Meter (PAM). The pressure application device is applied to the knee joint of the animal and the threshold is measured by animal's limb withdrawal as a result of pain. Pain response is represented in gram force (gF). PAM measurement is carried out weekly once throughout the experimental period on surviving animals. PAM measurement is done in both the knee joints and an average of two values is calculated for further analysis. Mean PAM value throughout the experimental period for each group is calculated and represented in tabular and graphical form (Tables 3 and 4, FIGS. 2 and 3).

TABLE 3
Effect of treatment on pain response (PAM Value) (Unit: Gram Force; Mean ±
SD)

| | | | | | MIA + | | | | | |
|---|---|---|---|---|---|---|---|---|---|---|
| | Saline | | MIA + | | P-A + | | MIA + HA + | | MIA + HA + | |
| | Control | | P-A | | HA | | BMMSC-L | | BMMSC-H | |
| | Mean | | Mean | | Mean | | Mean | | Mean | |
| Week | (gF) | SD | (gF) | SD | (gF) | SD | (gF) | SD | (gF) | SD |
| Week-0 | 870.4 | 209.4 | 480.4^{#} | 80.0 | 481.8 | 77.0 | 480.6 | 80.8 | 481.4 | 79.4 |
| Week-1 | 673.9 | 224.2 | 421.4^{#} | 108.8 | 492.6 | 105.0 | 450.2 | 96.2 | 463.5 | 109.6 |
| Week-2 | 715.4 | 193.7 | 431.8^{#} | 96.5 | 410.6 | 100.7 | $$533.6* | 61.2 | $$528.0 | 129.4 |
| Week-3 | 822.6 | 171.3 | 454.0^{#} | 69.4 | 568.8** | 107.2 | 644.4*** | 78.9 | $677.3*** | 107.9 |
| Week-4 | 905.5 | 127.0 | 491.0^{#} | 81.9 | 549.0 | 101.9 | 635.2*** | 70.9 | $$664.9*** | 80.0 |
| Week-5 | 849.7 | 125.8 | 508.0^{#} | 69.1 | 576.0 | 61.4 | $685.4*** | 56.0 | $685.4*** | 69.3 |
| Week-6 | 873.8 | 129.7 | 487.3^{#} | 64.4 | 546.7 | 52.4 | $659.8*** | 70.2 | $$681.7*** | 91.7 |
| Week-7 | 913.2 | 146.5 | 505.4^{#} | 83.9 | 554.9 | 55.2 | 653.9*** | 53.3 | $673.4*** | 83.4 |
| Week-8 | 1036.7 | 93.0 | 515.7^{#} | 95.2 | 539.5 | 47.7 | $$666.3*** | 76.2 | $$$716.3*** | 82.5 |
| Week-9 | 1042.7 | 59.9 | 588.1^{#} | 71.1 | 623.6 | 76.3 | 655.3 | 103.2 | 745.8* | 49.6 |
| Week- | 1033.3 | 96.1 | 580.3^{#} | 58.0 | 658.5 | 36.9 | 688.4 | 76.9 | 752.3* | 58.5 |
| 10 | | | | | | | | | | |
| Week- | 1095.2 | 97.3 | 617.5^{#} | 29.8 | 616.9 | 85.8 | 678.5 | 67.5 | 759.0 | 56.6 |
| 12 | | | | | | | | | | |

Each column represents mean ± SD. Data is statistically analyzed by Two way ANOVA followed by Bonferroni posttests.
Saline Control Vs MIA + P-A ^{#}p < 0.001
MIA + P-A Vs other treatment groups *p < 0.05; **p < 0.01 and ***p < 0.001
MIA + P-A + HA Vs BMMSC treatment groups $p < 0.05; $$p < 0.01 and $$$p < 0.001

The PAM score results demonstrated an increase in pain threshold in both the cell treated groups as compared to the HA group till end of the study. This increase is found to be statistically significant up to about 8 weeks of treatment. The mean PAM score after about 8 weeks follow-up for the HA treated group is about 540 g F as compared to the PAM score recorded for the BMMSC high dose group which is about 716 g F (p<0.001), and for the BMMSC low dose group which is about 666 g F (p<0.01). In terms of pain recovery, both the BMMSC treated groups recorded increased pain recovery from about week 3 onwards as compared to the HA treated group, and this recovery is maintained till the end of the study period (from about 8.8% to about 36.6% at the end of about 12 weeks). The results indicate the advantage of using BM-MSC in the management of Osteoarthritis pain

TABLE 4
Effect of treatment on overall pain response
(PAM Value) (Unit: Gram Force; Mean ± SD)

| Treatment | Mean | SD |
|---|---|---|
| Saline Control | 902.71 | 131.5 |
| MIA + P-A | 506.74 | 61.3 |
| MIA + P-A + HA | 551.56 | 67.5 |
| MIA + HA + | 619.29* | 82.6 |
| BMMSC - L | | |
| MIA + HA + | 652.42** | 103.4 |
| BMMSC - H | | |

Each column represents Mean ± SD.
Data is statistically analyzed by one way ANOVA followed by Bonferroni's Multiple Comparison Test.
MIA + P-A vs other treatment groups,
*P < 0.05 and
**P < 0.01

### Example 1.2

### Gross Pathology of Knee Joint

Gross appearance of knee articular cartilage of femoral side of the right knee joint that is extracted from the animals at each sacrifice time points are photographed using dissecting microscope.

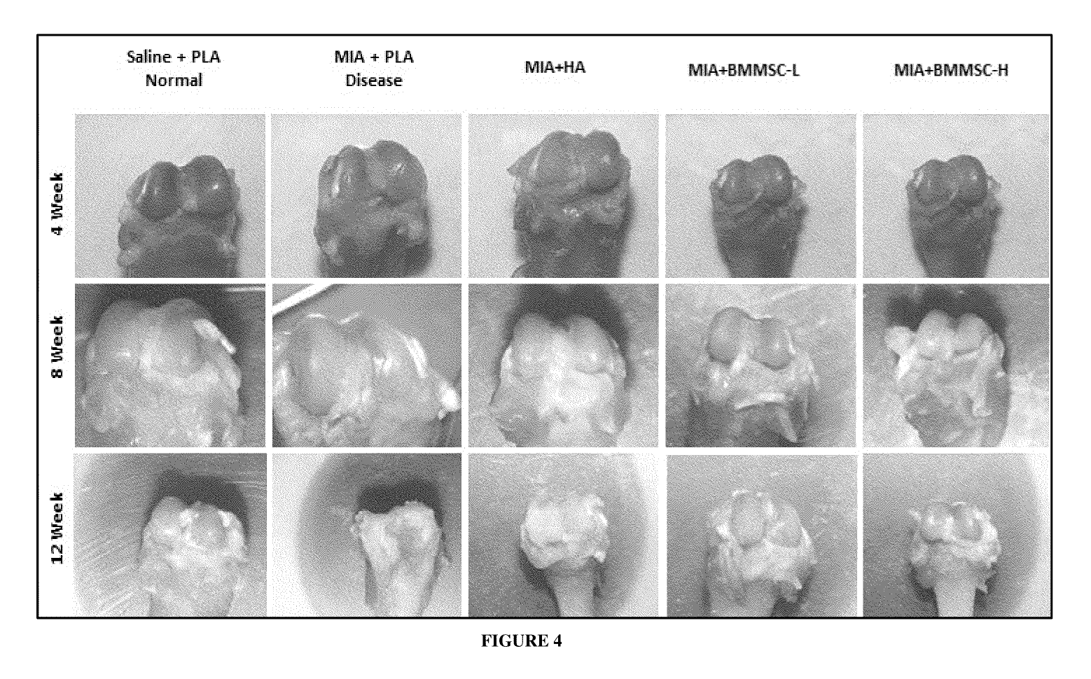

The femoral condyle surface of right knee joint is evaluated for surface abrasion and damage which is evident from surface roughness compared to the smooth, glassy appearance in normal control. Photographs of MIA induced OA in right knee joint of all the experimental group animals clearly represent the gross effect of test item (FIG. 4) and are scored (tables 5, 6, 7). No cartilage damage is found in saline+P-A (Plasmalyte A) injected group animals (G1) at all the termination time points. The cartilage layer of condyl surface of femur bone is free from degradation. However, in MIA+P-A injected group animals (G2), maximum loss and or degradation of cartilage layer on the condyl surface of femur bone is seen. At about 4 week termination time point, two animals out of three showed damage in the cartilage surface. Cartilage damage is seen in all the six animals terminated at about 8 week time point. Knee joints of about 12 week termination time point showed cartilage damage in five out of six animals.

MIA+P-A+HA injected group animals (G3) showed loss and or degradation of cartilage layer on the condyl surface of femur bone. At about 4 week termination time point, two animals out of three showed damage in the cartilage surface. Cartilage damage is seen in three animals out of six at 8 and 12 week termination time points.

MIA+P-A+BMMSC-H injected group animals (G4) showed no degradation of cartilage layer on the condyl surface of femur bone at about 4 week termination time point. Cartilage damage is seen in three out of six animals terminated at 8 week time point. Knee joints of about 12 week termination time point showed cartilage damage in one out of six animals.

MIA+P-A+BMMSC-L injected group animals (G5) showed no degradation of cartilage layer on the condyl surface of femur bone in one animal at about 4 week termination time point. Cartilage damage is seen in one out of six animals terminated at 8 week time point. Knee joints of 12 week termination time point indicated three out of six animals are found with damage in the cartilage.

Findings of gross observation of knee joint at various terminal point i.e. about 4 week, about 8 week and about 12 week indicates maximum osteoarthritis positive animals in MIA+P-A injected animals.

Cartilage damage is seen physically in 13 out of 15 assigned animals of group G2 (MIA+P-A) which results in about 86.7% Osteoarthritic animals. MIA+P-A+HA injected animals results in about 53.3% osteoarthritic animals in which 8 out of 15 animals is found with cartilage damage. MIA+HA+BMMSC-L treated animals showed about 40% osteoarthritic animals where, 6 out of 15 animals are found with damaged knee joint cartilage. Whereas MIA+HA+BMMSC-H treated animals showed about 26.7% osteoarthritic animals where, 4 out of 15 animals are found with cartilage damage. No visible tumor seen in gross observation of knee joints of all the treatment groups. Eleven animals in this group are found to be free from Osteoarthritic cartilage damage (Table-8)

TABLE 5
Gross pathology of knee joint cartilage (Week-4)

| | | | | % of | % of |
|---|---|---|---|---|---|
| | | Animals | Animals | Positive | Negative |
| | Number | with | with no | Oste- | Oste- |
| | of | Cartilage | Cartilage | oarthritic | oarthritic |
| Treatment | Animals | Damage | Damage | Animals | Animals |
| Saline + P-A | 2 | 0 | 2 | NA | NA |
| MIA + P-A | 3 | 2 | 1 | 66.7 | 33.3 |
| MIA + P-A + | 3 | 2 | 1 | 66.7 | 33.3 |
| HA | | | | | |
| MIA + HA + | 3 | 2 | 1 | 66.7 | 33.3 |
| BMMSC-L | | | | | |
| MIA + HA + | 3 | 0 | 3*^{#@} | 0 | 100 |
| BMMSC-H | | | | | |

^{#}Chi squre statistic is 3. The P value is 0.083265. This result is significant at p < 0.10 when compared to MIA + P-A + HA
*Chi squre statistic is 3. The P value is 0.083265. This result is significant at p < 0.10 when compared to MIA + P-A
^{@}Z Test: Z score is −1.7321. The P value is 0.04182; The result significant at p < 0.05 when compared to MIA + P-A + HA

TABLE 6
Gross pathology of knee joint cartilage (Week-8)

| | | | Animals | % of | % of |
|---|---|---|---|---|---|
| | | Animals | with | Positive | Negative |
| | Number | with | no | Oste- | Oste- |
| | of | Cartilage | Cartilage | oarthritic | oarthritic |
| Treatment | Animals | Damage | Damage | Animals | Animals |
| Saline + P-A | 2 | 0 | 2 | NA | NA |
| MIA + P-A | 6 | 6 | 0 | 100 | 0 |
| MIA + P-A + | 6 | 3 | 3** | 50 | 50 |
| HA | | | | | |
| MIA + HA + | 6 | 1 | 5*** | 16.7 | 83.3 |
| BMMSC-L | | | | | |
| MIA + HA + | 6 | 3 | 3** | 50 | 50 |
| BMMSC-H | | | | | |

**Chi squre statistic is 4. The P value is 0.0455. This result is significant at p < 0.05 when compare to MIA + P-A
***Chi squre statistic is 8.5714. The P value is 0.003415. This result is significant at p < 0.01 when compare to MIA + P-A

TABLE 7
Gross pathology of knee joint cartilage (Week-12)

| | | | Animals | % of | % of |
|---|---|---|---|---|---|
| | | Animals | with | Positive | Negative |
| | Number | with | no | Oste- | Oste- |
| | of | Cartilage | Cartilage | oarthritic | oarthritic |
| Treatment | Animals | Damage | Damage | Animals | Animals |
| Saline + P-A | 2 | 0 | 2 | NA | NA |
| MIA + P-A | 6 | 5 | 1 | 83.3 | 16.7 |
| MIA + P-A + | 6 | 3 | 3 | 50 | 50 |
| HA | | | | | |
| MIA + HA + | 6 | 3 | 3 | 50 | 50 |
| BMMSC-L | | | | | |
| MIA + HA + | 6 | 1 | 5** | 16.7 | 83.3 |
| BMMSC-H | | | | | |

**Chi squre statistic is 5.3333. The P value is 0.020921. This result is significant at p < 0.05 when compare to MIA + P-A

TABLE 8
Summary of gross pathology of knee joint cartilage (Week-4, Week-8 &
Week-12)

| | | | MIA + | MIA + | |
|---|---|---|---|---|---|
| | Saline + | MIA + | P-A + | HA + | MIA + HA + |
| Treatment | P-A | P-A | HA | BMMSC-L | BMMSC-H |
| Total No. of | 6 | 15 | 15 | 15 | 15 |
| animals | | | | | |
| Osteoarthritis | NA | 13 | 8 | 6 | 4 |
| Positive | | | | | |
| Osteoarthritis | NA | 2 | 7** | 9**** | 11***^{@} |
| Negative | | | | | |
| % of Positive | NA | 86.7 | 53.3 | 40 | 26.7 |
| Osteoarthritic | | | | | |
| Animals | | | | | |
| % of Negative | NA | 13.3 | 46.7 | 60 | 73.3 |
| Osteoarthritic | | | | | |
| Animals | | | | | |

**Chi squre statistic is 3.9683. The P value is 0.046366. This result is significant at p < 0.05 when compared to MIA + P-A
***Chi squre statistic is 10.9955. The P value is 0.000913. This result is significant at p < 0.01 when compared to MIA + P-A
****Chi squre statistic is 7.0335. The P value is 0.008. This result is significant at p < 0.01 when compared to MIA + P-A
^{@}Z Test: Z score is −1.4907. The P value is 0.06811; The result significant at p < 0.1 when compared to MIA + P-A + HA

### Example 1.3

### Hispathological Observation and Oarsi Grading

The right knee joint of the animals is excised and the bone is then subjected for decalcification. Initially the knee joint bone is fixed overnight in about 10% formalin solution. Bone is then transferred individually to the decalcifying agent (about 10% EDTA). The decalcifying agent is changed about weekly twice for about two weeks until traces of calcium precipitate are not seen.

After ensuring the complete decalcification by checking the calcium content qualitatively, the knee joint bones are subjected to histopathological process.

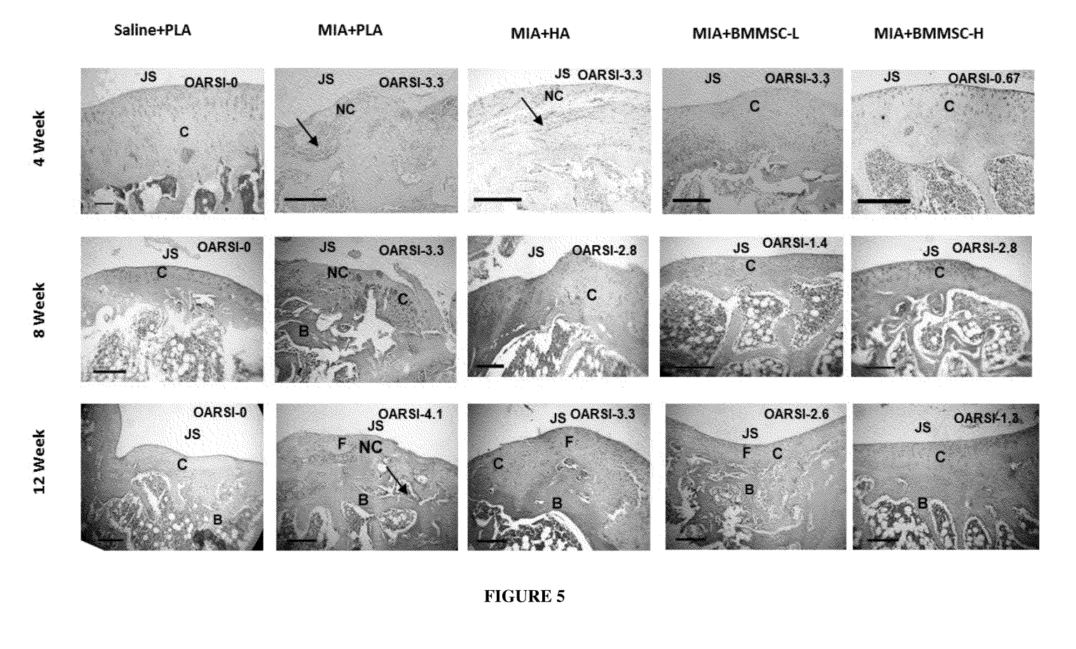

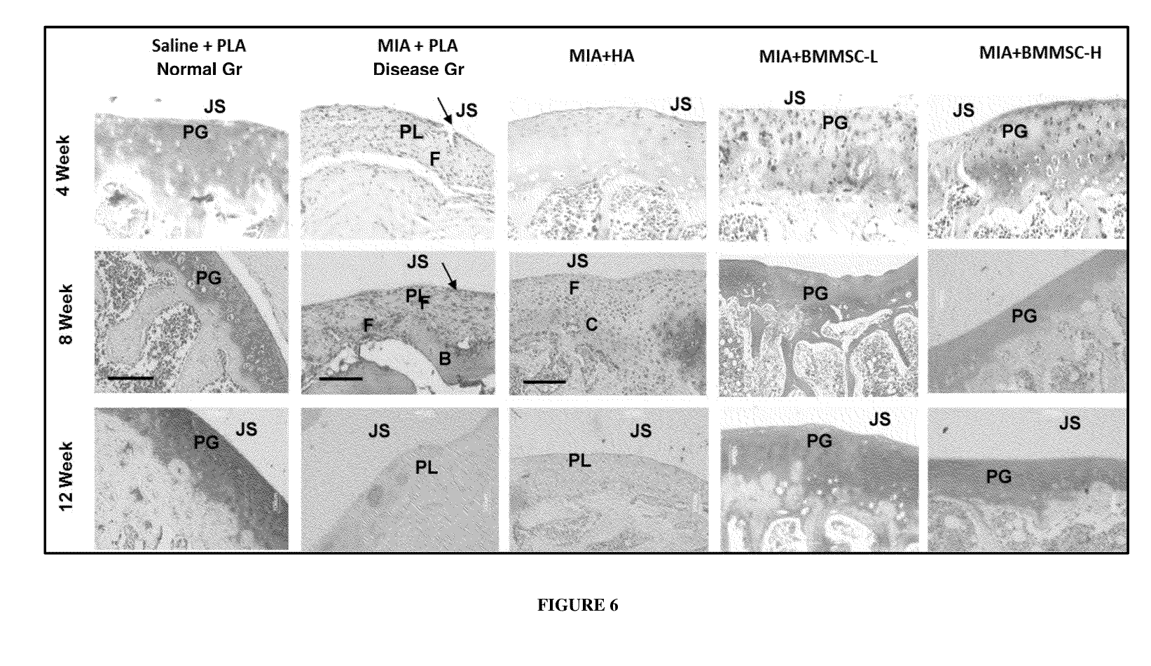

Hematoxylin & Eosin (H&E), staining is performed for grade and stage assessment (Grade 0 to 6 & stage 0 to 4) as per OARSI (Osteoarthritis Research Society International) grading to find the severity of Osteoarthritic disease mentioned in the below tables (FIG. 5). Histological observation is done for cartilage cap damage and graded which is equivalent to OARSI grading as per standard procedure. Safranin O and fast green staining is also carried out (FIG. 6). Presence of BMMSC cell into the joint space is also confirmed by HNA-Immunoflurosence assay.

The Histological findings of 4^{th} week & 12^{th} week termination time point showed preservation of articular cartilage in the MIA+P-A+BMMSC-H Group, while at 8^{th} week termination time point MIA+P-A+BMMSC-L Group showed protection from cartilage damage when compared to MIA+P-A+BMMSC-H Group based on the OARSI grading system. The total Eq. OARSI Grade in MIA treated animal group is about 3.61. While the total Eq. OARSI Grade in MIA+P-A+HA and MIA+HA+BMMSC-L treated animal group is more than about 2.50. However the total Eq. OARSI Grade in MIA+HA+BMMSC-H treated group animals is about 1.61. The MIA+HA+BMMSC-H treated group animals showed less OARSI Grade when compared to other treatment groups. (Table 9 and FIG. 5)

TABLE 9
Histological Observations of knee joint and Equivalent OARSI Grading

| | | | | | | % Decrease |
|---|---|---|---|---|---|---|
| | | | | | Total Eq. | in Eq. |
| Group | | Average Eq. OARSI Grade | | | OARSI | OARSI |
| No. | Treatment | 4 Week | 8 Week | 12 Week | Grade | Grade |
| G1 | Saline + P-A | 0 | 0 | 0 | 0.00 | NA |
| G2 | MIA + P-A | 3.33 ± 2.89 | 3.33 ± 2.07 | 4.17 ± 2.04 | 3.61 ± 2.33 | 0.00 |
| G3 | MIA + P-A + | 3.33 ± 2.89 | 2.83 ± 2.32 | 3.33 ± 2.58 | 3.17 ± 2.60 | 12.31 |
| | HA | | | | | |
| G5 | MIA + HA + | 3.33 ± 2.89 | 1.40 ± 2.19 | 3.33 ± 2.58 | 2.69 ± 2.55 | 25.54 |
| | BMMSC-L | | | | | |
| G4 | MIA + HA + | 0.67 ± 1.15 | 2.83 ± 2.48 | 1.33 ± 2.16 | 1.61 ± 1.93 | 55.38 |
| | BMMSC-H | | | | | |

About 4^{th} week histological findings indicates a much lesser degree of cartilage damage in the MIA+HA+BMMSC—High dose group as compared to the MIA+HA+BMMSC—Low dose, MIA+P-A+HA & MIA+P-A groups. The histological features seen at about 8^{th} week indicates much less cartilage damage in the MIA+H-A+BMMSC Low dose group as compared to MIA+H-A+BMMSC—High dose, MIA+P-A+HA and MIA+P-A groups. Histological examination at about 12 week showed significantly less cartilage damage in MIA+HA+BMMSC—High dose group as compared to MIA+HA+BMMSC—low dose, MIA+P-A+HA and MIA+P-A groups.

### Safranin ‘O’ Staining:

Safranin ‘O’ stained sections of about 4^{th}, 8^{th} and 12^{th} week showed good correlation with the histological features seen in H & E image as shown in FIG. 5. Thus, the sections with extensive cartilage damage showed loss of Safranin ‘O’ staining along the articular cartilage. BMMSC injected knee joint sections are found to retain Safranin “O” staining in all the termination points; hence this indicates prevention of cartilage damage and probable role of BMMSC treatment in cartilage regeneration, refer FIG. 6 and table 10.

TABLE 10
Summary of histology of Safrannin O staining

| Treatment | 4 week | 8 week | 12 week |
|---|---|---|---|
| Saline + P-A | Cartilage preserved | Cartilage preserved | Cartilage preserved |
| | with good S-O | with good S-O | with good S-O staining |
| | staining | staining | |
| MIA + P-A | Absence of S-O | Absence of S-O | Absence of S-O |
| | staining | staining, Articular | staining, Articular |
| | | surface covered by | surface covered by |
| | | fibrous tissue | fibrous tissue |
| MIA + P-A + HA | Cartilage preserved | Cartilage preserved | Absence of S-O |
| | with good S-O | with good S-O | staining, Articular |
| | staining | staining | surface covered by |
| | | | fibrous tissue |
| MIA + BMMSC- | Cartilage preserved | Cartilage preserved | Cartilage preserved |
| L + HA | with good S-O | with good S-O | with good S-O staining |
| | staining | staining | |
| MIA + BMMSC- | Cartilage preserved | Cartilage preserved | Cartilage preserved |
| H + HA | with good S-O | with good S-O | with good S-O staining |
| | staining | staining | |

Based on the gross and histological assessment, there is a significant prevention of cartilage damage by BMMSC in a dose dependent manner indicating the active contribution of BMMSC in modulating the osteoarthritis disease progression.

### Immunofluorescence Staining

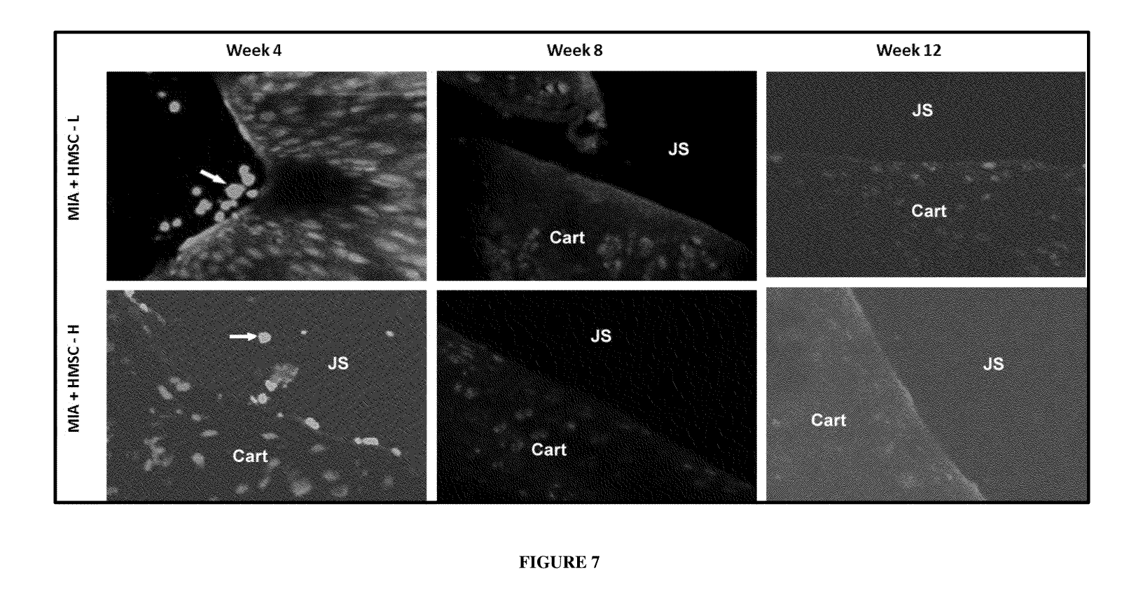

Immunofluorescence staining for human nuclear antigen (HNA) is carried out to trace the survival and migration pattern of the BMMSCs in the knee joints. The HNA labeled cells are found throughout the osteoarthritic cartilage at about 4 weeks post cell transplantation. Presence of HNA staining at the cartilage indicates survival and engraftment of the MSC in the OA joint (FIG. 7).

### Example 1.4

### Estimation of Cartilage Specific Rat Collagen Type II Protein

Left knee is dissected above ankle joint from the animals at each sacrifice points and subjected for homogenization to extract protein contents. The bony tissue from left knee is broken into pieces and mixed with RIPA extraction buffer. Tissues are then homogenized. The sample is rinsed with ice-cold PBS and transferred to ice-cold lysis buffer (RIPA Buffer). Cellular debris is removed by centrifugation and the supernatant of homogenate is subjected to biomarker analysis by ELISA as per manufacturer's (USCN, Life sciences Inc, China) instructions provided in the manual.

Estimation of specific rat biomarkers demonstrated significant increase in the content of rat type II collagen (p<0.01) in BMSSC treated animals as compared to the HA treated animals at about week 4 which adds structure and strength to connective tissues in the cartilage. The results indicate that the single intra articular injection of BMMSC improved the cartilage regeneration by increasing the cartilage ECM collagen Type II (FIG. 8 and table 11).

TABLE 11
Level of human collagen type-II(Unit: ng/knee; Mean ± SEM)

4 Week

| Treatment | Mean | SEM |
|---|---|---|
| Saline + PlasmaLyte A | −4.0 | 0.4 |
| MIA + PlasmaLyte A | 1.7 | 1.1 |
| MIA + PlasmaLyte A + HA | −0.9 | 1.5 |
| MIA + BMMSC-H + HA | −0.3 | 1.5 |
| MIA + BMMSC-L + HA | −1.4 | 0.6 |

### Example 2

### Clinical Study

A randomized, double blind, multicentric, placebo controlled, phase-II study is conducted in about 5 hospitals across India for assessing the safety and efficacy of intra-articular administration of composition comprising ex vivo cultured adult allogeneic pooled mesenchymal stromal cells derived from Bone marrow (BM-MSCs), in patients with Osteoarthritis of knee joint selected for the trial based on the exclusion and inclusion criteria.

About sixty patients in the age group of about 40-60 years diagnosed with grade 2 to 3 Osteoarthritis based on the Kellgren and Lawrence radiographic criteria, as depicted in the below table 12, are included in the study.

TABLE 12
Kellgren and Lawrence radiographic criteria

| Kellgren & | |
|---|---|
| Lawrence grade | Kellgren & Lawrence definition |
| Grade 1 ‘Doubtful’ | Minute osteophyte, doubtful significance |
| Grade 2 ‘Minimal’ | Definite osteophyte, unimpaired joint space |
| Grade 3 ‘Moderate’ | Moderate dimunition of joint space |
| Grade 4 ‘Severe’ | Joint space greatly impaired with sclerosis of |
| | subchondral bone |

The patients are randomized to receive a single intra articular injection of composition comprising pooled BM-MSCs (about 25/50/75/150 million cells) followed by about 2 ml of hyaluronic acid. Following the injection, patients are followed up at period intervals till the end of about 6 months. All patients are further followed up till the end of about 2 years.

Out of the sixty patients enrolled in the study, about fifteen patients are randomized to stem cell arm and placebo arm in a ratio of about 2:1 at each dose level. Thus, about 10 patients receive composition comprising pooled MSCs and about 5 patients receive placebo at each dose level.

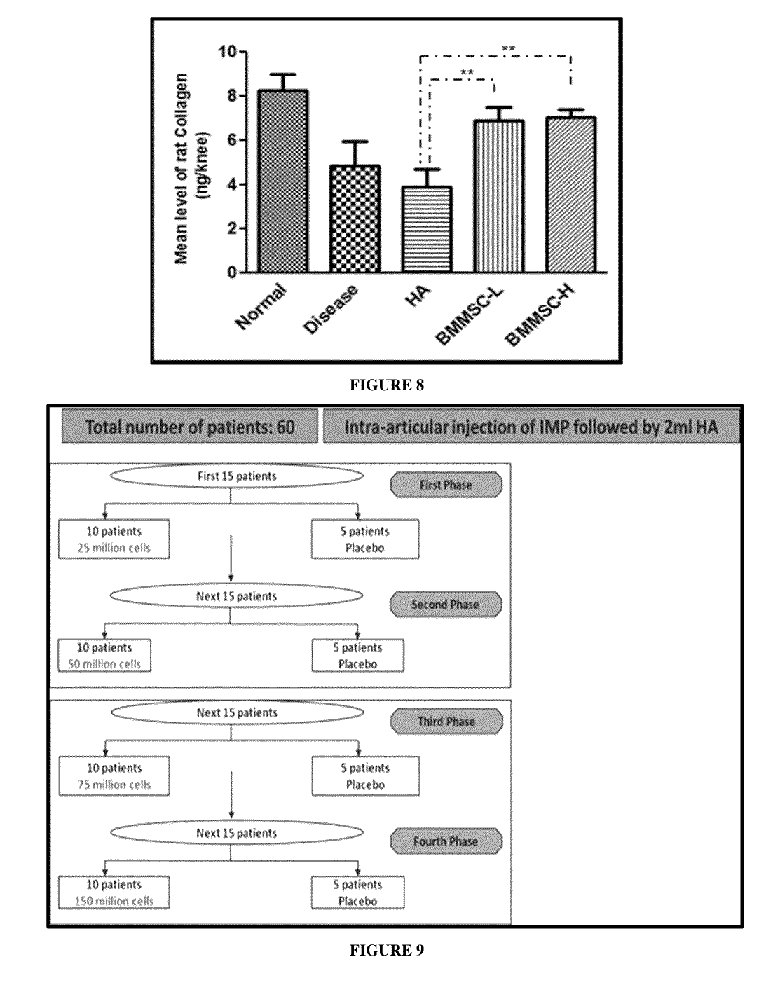

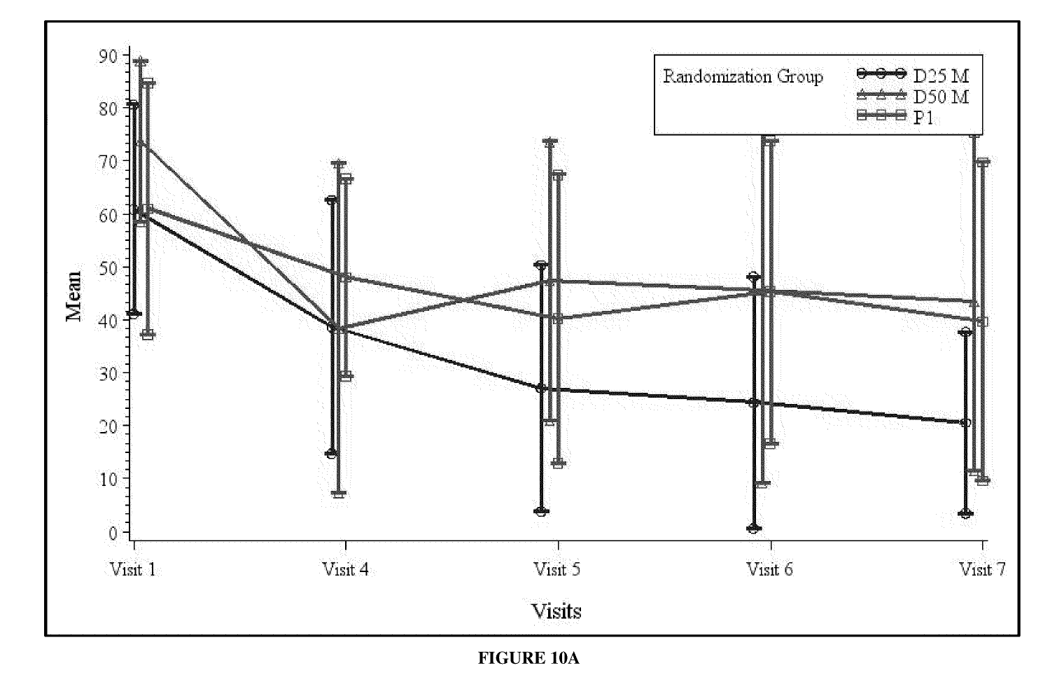

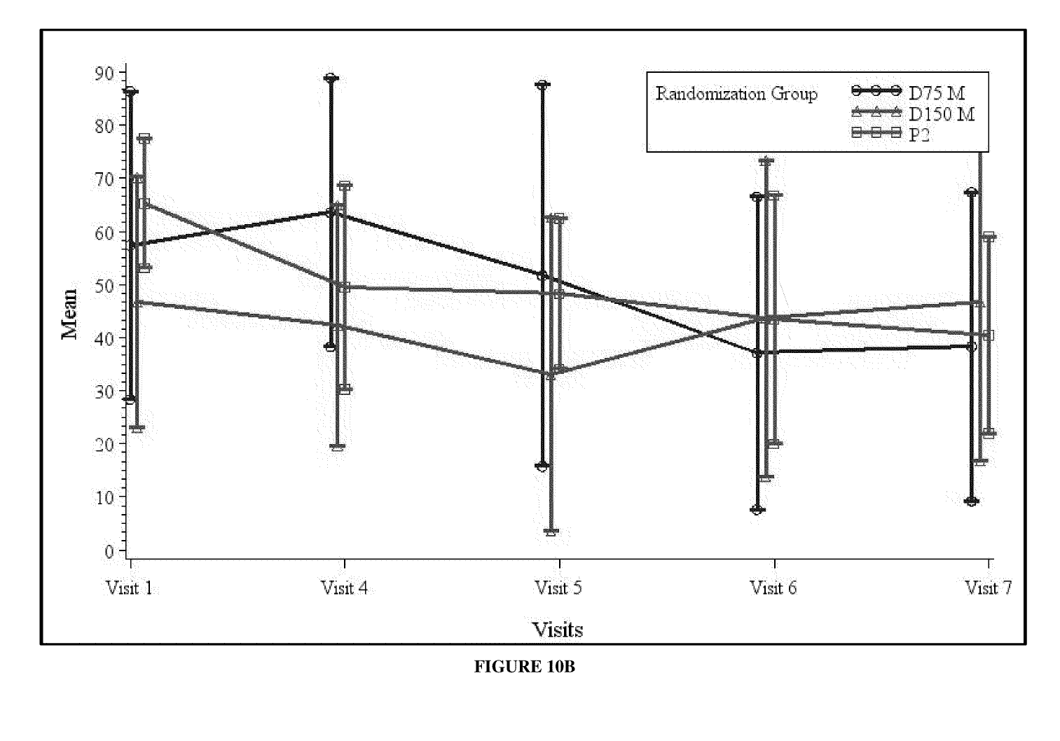

The doses used in this study are about 25 million, about 50 million, about 75 million- and about 150 million-pooled BM-MSCs. Cohort 1 includes patients administered with pooled BM-MSCs at a dose of about 25 million cells and about 50 million cells and the placebo P1 corresponding to about 25 million and about 50 million cells groups. Cohort 2 includes patients administered with pooled BM-MSCs at a dose of about 75 million cells and about 150 million cells and the placebo P2 corresponding to about 75 million and about 150 million cells groups. The cells are suspended in about 2-4 ml of PlasmaLyte A and injected followed by about 2 ml Hyaluronan injection. Twenty patients in the placebo arm receive only intra-articular injection of about 2-4 ml PlasmaLyte A followed by about 2 ml Hyaluronan. The Study design for the trial is represented by FIG. 9.

TABLE 13
Baseline characteristics of the study groups

| | Age (yrs) | Weight (kg) | | | |
|---|---|---|---|---|---|
| | (mean ± SD) | (mean ± SD) | Sex (M:F) | KL grade II | KL grade III |
| 25M | About 58.1 ± 8.2 | About 73.1 ± 15.8 | About 3:7 | 4 | 6 |
| 50M | About 57.3 ± 9.4 | About 69 ± 14.6 | About 2:8 | 1 | 9 |
| P1 | About 54.9 ± 8.2 | About 66.1 ± 7.6 | About 0:10 | 3 | 7 |
| 75M | About 55 ± 6.7 | About 71.3 ± 9.0 | About 2:8 | 1 | 9 |
| 150M | About 54 ± 6.7 | About 66 ± 9.1 | About 5:5 | 3 | 7 |
| P2 | About 56.7 ± 5.1 | About 66.9 ± 8.5 | About 3:7 | 2 | 8 |

The above table 13 shows that baseline characteristics are comparable across all groups. P1 indicates the Placebo arm for cohort 1 patients (about 25M and about 50M), wherein the total volume injected is about 2 ml of PlasmaLyteA. P2 indicates the Placebo arm for cohort 2 patients (about 75M and about 150M), wherein the total volume of about 4 ml of PlasmaLyteA is injected.

The primary end points of the study are safety and tolerability, assessed by adverse events (table 14), hematology, biochemical parameters and ECG. The secondary endpoints of the study are change from baseline in the WOMAC OA (Western Ontario and McMaster Universities Osteoarthritis) Index-pain subscale score, WOMAC Osteoarthritis stiffness Index, WOMAC Osteoarthritis Composite Index, ICOAP (Intermittent and Constant Osteoarthritis Pain), patient's assessment of Osteoarthritis Pain by VAS (Visual analog scale) and WORMS (Whole Organ Magnetic Resonance Imaging Score) scoring system using MRI of the knee and assessment of cartilage thickness using MRI at different points of the knee joint. Evaluation of these parameters establishes efficacy of the pooled BM-MSCs.

TABLE 14
Overall Summary of Adverse Events (Safety Population)

| | D25M | D50M | P1 | D75M | D150M | P2 |
|---|---|---|---|---|---|---|
| | (N = 10) | (N = 10) | (N = 10) | (N = 10) | (N = 10) | (N = 10) |
| | No. of | No. of | No. of | No. of | No. of | No. of |
| | Events | Events | Events | Events | Events | Events |
| CATEGORY | (n) | (n) | (n) | (n) | (n) | (n) |
| Any AE | 24 (7) | 13 (7) | 21 (7) | 17 (7) | 11 (6) | 11 (6) |
| Any SAE | 1 (1) | 0 | 2 (1) | 0 | 1 (1) | 2 (2) |
| Any related AE | 1 (1) | 2 (1) | 0 | 8 (4) | 7 (5) | 1 (1) |
| Any AE with outcome = | 0 | 0 | 0 | 0 | 0 | 0 |
| death | | | | | | |
| Any AE leading to | 0 | 0 | 0 | 0 | 0 | 0 |
| discontinuation of | | | | | | |
| treatment | | | | | | |
| Any SAE leading to death | 0 | 0 | 0 | 0 | 0 | 0 |
| (including events with | | | | | | |
| outcome = death) | | | | | | |
| Any SAE with outcome | 1 (1) | 0 | 2 (1) | 0 | 1 (1) | 2 (2) |
| other than death | | | | | | |

N = Total number of patients in the specified group
n = Number of patients with at least one AE during the study.
D25M = 25 million MSCs,
D50M = 50 million MSCs,
P1 = Placebo 2 ml,
D75M = 75 million MSCs,
D150M = 150 million MSCs,
P2 = Placebo 4 ml.
For counting number of events, AEs reported within each system organ class/preferred term more than once due to change in severity is counted only once.
Source Listing: Adverse Events.

### Assessment of Osteoarthritis Pain by Visual Analog Scale

FIGS. 10A and 10B depict a graphical representation of a visual analogue scale (VAS) in the study groups. The X-axis of the figures represents different visits in which the evaluations of the patients for the respective parameters are done. Visit 1: baseline, Visit 4: 1 month follow-up, Visit 5: 3 months follow-up, Visit 6: 6 months follow-up and Visit 7: 1 year follow-up. The Y-axis represents score of the evaluated parameter. The parameter evaluated is a measure of the subjective assessment of pain using a VAS (100 mm long) in which zero represents no pain and 100 mm represents worst pain. Patients are asked to Place an “X” mark on the line to indicate how bad he/she felt the pain on the day of evaluation in relation to the two extremes. Different lines represent the different groups in the study. 25M: about 25 million cells, 50M: about 50 million cells, P1: placebo corresponding to about 25M and about 50M groups; 75M: about 75 million cells, 150M: about 150 million cells, P2: placebo corresponding to about 75M and about 150M groups. Reduction of VAS values in the follow-up compared to baseline indicates improvement in the pain (or relief of pain).

The mean (SD) pain VAS score for 25M group, 50M group and P1 is about 60.9 (19.7), about 73.7 (15.2) and about 61.0 (23.8) respectively at baseline visit. The mean (SD) pain VAS score for 25M group, 50M group and P1 is about 38.7 (24.0), about 38.4 (31.2) and about 48.0 (18.6) respectively at about 1 month (visit 4); is about 27.1 (23.3), about 47.3 (26.4) and about 40.2 (27.2) respectively at about 3 months (visit 5); is about 24.4 (23.8), about 45.6 (36.4) and about 45.3 (28.6) respectively at about 6 months (visit 6); and is about 20.6 (17.1), about 43.4 (32.0) and about 39.7 (30.1) respectively at about 12 months (visit 7). The VAS [osteoarthritis-Pain Visual analog scale (VAS)] is improved from about 60.9 mm at baseline to about 20.6 mm (about 66% reduction) in about 1 year in 25 million dose compared to about 61 mm at baseline to about 39.7 mm in about 1 year (about 35% improvement) in placebo arm (FIG. 20A). Within cohort 1 (25M, 50M and P1), there is a consistent decrease in VAS score in 25M dose group and 50M dose group or placebo groups. The difference is seen starting from about 3 months (visit 5) till about 12 months (visit 7).

The mean (SD) VAS score for D75M group, D150M group and P2 is about 57.4 (29.0), about 46.6 (23.6) and about 65.3 (12.2) respectively at baseline visit. The mean (SD) VAS score for D75M group, D150M group and P2 is about 63.6 (25.3), about 42.3 (22.6) and about 49.5 (19.2) respectively at about 1 month (visit 4); is about 51.7 (35.9), about 33.1 (29.5) and about 48.3 (14.1) respectively at about 3 months (visit 5); is about 37.1 (29.4), about 43.6 (29.8) and about 43.4 (23.4) respectively at about 6 months (visit 6); and is about 38.3 (29.1), about 46.6 (29.9) and about 40.5 (18.6) respectively at 12 months (visit 7). Within cohort 2 (75M, 150M and P2), there is no remarkable difference in the improvement in any one particular group when compared to other groups.

### Western Ontario and McMaster Universities Osteoarthritis (WOMAC OA) Index

The Western Ontario and McMaster Universities Arthritis Index (WOMAC) is a widely used, proprietary set of standardized questionnaires used by health professionals to evaluate the condition of patients with osteoarthritis of the knee and hip, including pain, stiffness, and physical functioning of the joints. The WOMAC measures five items for pain (during walking, using stairs, in bed, sitting or lying, and standing) (score range 0-500), two for stiffness (after first waking and later in the day) (score range 0-200), and 17 for functional limitation (score range 0-1700). Physical functioning questions cover everyday activities such as stair use, standing up from a sitting or lying position, standing, bending, walking, getting in and out of a car, shopping, putting on or taking off socks, lying in bed, getting in or out of a bath, sitting, and heavy and light household duties. The total WOMAC is calculated as sum total score of pain, stiffness and physical function score (score range 0-2400). The WOMAC scoring is performed at baseline and after about one month (visit 4), after about 3 months (visit 5), after about 6 months (visit 6) and after about 12 months (visit 7) of administration of the cell composition/IMP injection.

### WOMAC Pain Index:

The WOMAC pain index scores across all the treatment groups are comparable at baseline [tables 15(a) and (b)]. The baseline WOMAC pain index scores ranged between about 248.8 in P1 group to about 298.2 in the D50M group.

WOMAC pain index scores decreased substantially over the study period for all the treatment groups. The maximum mean and percentage change from baseline in the scores across the treatment groups is seen in the D25M group at about 6 months (visit 6) and about 12 months (visit 7).

In D25M group, the mean (SD) WOMAC pain index score changed from about 256.3 (114.3) at baseline to about 134.7 (121.3) at about 6 months (visit 6) and about 111.2 (95.9) at about 12 months (visit 7). The mean (SD) change from baseline is about 121.6 (130.0) at about 6 months (visit 6) (about 40.7%) and about 145.1 (105.2) at about 12 months (visit 7) (about 60.4%).

The mean (SD) score in D50M group is about 298.2 (103.2) at baseline, which changed to about 201.0 (125.2) at about 6 months (visit 6) and to about 224.1 (116.0) at about 12 months (visit 7). The mean (SD) change from baseline is about 97.2 (153.4) at about 6 months (visit 6) (18.4%) and about 74.1 (167.2) at about 12 months (visit 7) (6.7%).

The mean (SD) score in P1 group is about 248.8 (103.4) at baseline, which changed to about 187.5 (119.1) at about 6 months (visit 6) and to about 191.4 (163.7) at about 12 months (visit 7). The mean (SD) change from baseline is about 61.3 (107.3) at about 6 months (visit 6) (about 22.4%) and about 57.4 (151.3) at about 12 months (visit 7) (about 21.3%).

The mean (SD) score in D75M group is about 288.7 (114.9) at baseline, which changed to about 222.3 (120.7) at about 6 months (visit 6) and to about 168.2 (112.2) at about 12 months (visit 7). The mean (SD) change from baseline is about 66.4 (115.5) at about 6 months (visit 6) (about 12.4%) and about 100.1 (139.2) at about 12 months (visit 7) (about 20.5%). The mean (SD) score in D150M group is about 283.5 (99.9) at baseline, which changed to about 213.5 (179.5) at about 6 months (visit 6) and to about 235.2 (148.1) at about 12 months (visit 7).

The mean (SD) change from baseline is about 70.0 (156.1) at about 6 months (visit 6) (about 28.8%) and about 48.3 (111.0) at about 12 months (visit 7) (about 20.9%).

The mean (SD) score in P2 group is about 268.2 (about 72.2) at baseline, which changed to about 205.2 (117.5) at about 6 months (visit 6) and to about 228.6 (103.5) at about 12 months (visit 7). The mean (SD) change from baseline was about 63.0 (96.8) at 6 months (visit 6) (about 26.4%) and about 39.6 (84.8) at about 12 months (visit 7) (about 15.8%).

The Wilcoxon signed-rank test (within group analysis) at about 6 months (visit 6) (p value=0.0273) and about 12 months (visit 7) (p value=0.0020) showed a statistically significant improvement in WOMAC pain index score relative to the baseline for the D25M group. While D75M group showed a statistically significant improvement only at about 6 months (visit 6) (p value=0.0371) relative to the baseline.

However the ANOVA test showed no statistically significant difference when the mean change from baseline to 6 months (visit 6) [p value cohort 1: 0.5940, cohort 2: 0.3635] and 12 months (visit 7) [p value cohort 1: 0.9922, cohort 2: 0.4669] is compared within each cohort.

TABLE 15(a)
WOMAC pain index - Mean change from baseline

| | | | | | | | MEAN CHANGE FROM | | | |
|---|---|---|---|---|---|---|---|---|---|---|
| | | | POST BASELINE | | | | BASELINE | | | |
| | | | 6 Months | | 12 Months | | 6 Months | | 12 Months | |
| TREATMENT | BASELINE | | (Visit 6) | | (Visit 7) | | (Visit 6) | | (Visit 7) | |
| GROUPS | Mean | SD | Mean | SD | Mean | SD | Mean | SD | Mean | SD |
| D25M | 256.3 | 114.3 | 134.7 | 121.3 | 111.2 | 95.9 | −121.6 | 130.0 | −145.1 | 105.2 |
| D50M | 298.2 | 103.2 | 201.0 | 125.2 | 224.1 | 116.0 | −97.2 | 153.4 | −74.1 | 167.2 |
| P1 | 248.8 | 103.4 | 187.5 | 119.1 | 191.4 | 163.7 | −61.3 | 107.3 | −57.4 | 151.3 |
| D75M | 288.7 | 114.9 | 222.3 | 120.7 | 168.2 | 112.2 | −66.4 | 115.5 | −100.1 | 139.2 |
| D150M | 283.5 | 99.9 | 213.5 | 179.5 | 235.2 | 148.1 | −70.0 | 156.1 | −48.3 | 111.0 |
| P2 | 268.2 | 72.2 | 205.2 | 117.5 | 228.6 | 103.5 | −63.0 | 96.8 | −39.6 | 84.8 |

TABLE 15(b)
WOMAC pain index - Percentage change from Baseline

| | 6 MONTHS | | 12 MONTHS | |
|---|---|---|---|---|
| | (VISIT 6) | | (VISIT 7) | |
| TREATMENT GROUPS | Mean | SD | Mean | SD |
| D25M | −40.7 | 49.4 | −60.4 | 31.5 |
| D50M | −18.4 | 80.1 | −6.7 | 81.4 |
| P1 | −22.4 | 47.4 | −21.3 | 67.7 |
| D75M | −12.4 | 61.2 | −20.5 | 89.8 |
| D150M | −28.8 | 59.0 | −20.9 | 42.1 |
| P2 | −26.4 | 38.4 | −15.8 | 30.0 |

### WOMAC Stiffness Index:

The WOMAC stiffness index scores across all the treatment groups are comparable at baseline [tables 16(a) and (b)]. The baseline WOMAC stiffness index scores ranged between 102 in P1 group to 132.9 in the D75M group.

WOMAC stiffness index scores decreases substantially over the study period for all the treatment groups except for D50M group. The maximum mean and percentage change from baseline in the scores across the treatment groups is seen in the D150M and D25M group at about 6 months (visit 6) and in D25M and D75M at about 12 months (visit 7).

In D25M group, the mean (SD) WOMAC stiffness index score changed from about 120.6 (50.1) at baseline to about 72.7 (61.5) at about 6 months (visit 6) and about 51.0 (42.9) at about 12 months (visit 7). The mean (SD) change from baseline is about 47.9 (45.8) at about 6 months (visit 6) (about 44.1%) and about 69.6 (44.7) at 12 months (visit 7) (61.1%).

The mean (SD) score in D50M group is about 106.1 (45.2) at baseline, which changed to about 103.7 (55.2) at about 6 months (visit 6) and to about 110.6 (61.0) at about 12 months (visit 7). The mean (SD) change from baseline is about 2.4 (78.3) at about 6 months (visit 6) (increase of about 38.6%) and an increase of about 4.5 (87.2) at about 12 months (visit 7) (increase of about 52%).

The mean (SD) score in P1 group is about 102.0 (49.6) at baseline, which changed to about 71.2 (53.1) at about 6 months (visit 6) and to about 76.2 (65.7) at about 12 months (visit 7). The mean (SD) change from baseline is about 30.8 (29.8) at about 6 months (visit 6) (about 36.5%) and about 25.8 (53.4) at about 12 months (visit 7) (about 30.9%).

The mean (SD) score in D75M group is about 132.9 (41.9) at baseline, which changed to about 95.7 (53.1) at 6 months (visit 6) and to about 60.3 (38.4) at about 12 months (visit 7). The mean (SD) change from baseline is about 37.2 (52.1) at about 6 months (visit 6) (about 24.8%) and about 66.2 (49.5) at about 12 months (visit 7) (about 49.4%).

The mean (SD) score in D150M group is about 121.6 (35.0) at baseline, which changed to about 73.3 (68.4) at about 6 months (visit 6) and to about 101.3 (71.6) at about 12 months (visit 7). The mean (SD) change from baseline is about 48.3 (48.1) at about 6 months (visit 6) (46.4%) and about 20.3 (50.7) at about 12 months (visit 7) (about 23.5%).

The mean (SD) score in P2 group is about 113.9 (22.4) at baseline, which changed to about 83.0 (55.8) at about 6 months (visit 6) and to about 81.3 (37.7) at about 12 months (visit 7). The mean (SD) change from baseline is about 30.9 (42.6) at about 6 months (visit 6) (about 31.7%) and about 32.6 (27.4) at about 12 months (visit 7) (about 30.3%).

The Wilcoxon signed-rank test (within group analysis) showed a statistically significant improvement WOMAC stiffness index score relative to the baseline for the D25M group at about 6 months (visit 6) (p value=0.0098) and about 12 months (visit 7) (p value=0.0020), for P1 group only at about 6 months (visit 6) (p value=0.0137) relative to the baseline. The D150M and P2 groups showed a statistically significant improvement both at about 6 months (visit 6) [(p value=0.0195) and (p value=0.0488) respectively] and about 12 months (visit 7) [(p value=0.0039) and (p value=0.0039) respectively] relative to the baseline.

However the ANOVA test showed no statistically significant difference when the mean change from baseline to about 6 months (visit 6) [p value cohort 1: 0.1947, cohort 2: 0.7148] and about 12 months (visit 7) [p value cohort 1: 0.0504, cohort 2: 0.0808] is compared within each cohort.

A statistically significant difference (p value=0.0279) is observed when the mean differences of D25M and D50M groups are compared (using t-test). The D25M group showed a higher mean change compared to D50M group (D25M vs. D50M: −69.6 Vs 4.5). A statistically significant difference is observed when the mean percentage change from baseline are compared for the cohort treatment groups at about 12 months (p value=0.0372).

TABLE 16(a)
WOMAC stiffness index - Mean change from Baseline

| | | | Post Baseline | | | | Change from Baseline | | | |
|---|---|---|---|---|---|---|---|---|---|---|
| | | | 6 Months | | 12 Months | | 6 Months | | 12 Months | |
| Treatment | Baseline | | (Visit 6) | | (Visit 7) | | (Visit 6) | | (Visit 7) | |
| groups | Mean | SD | Mean | SD | Mean | SD | Mean | SD | Mean | SD |
| D25M | 120.6 | 50.1 | 72.7 | 61.5 | 51.0 | 42.9 | −47.9 | 45.8 | −69.6 | 44.7 |
| D50M | 106.1 | 45.2 | 103.7 | 55.2 | 110.6 | 61.0 | −2.4 | 78.3 | 4.5 | 87.2 |
| P1 | 102.0 | 49.6 | 71.2 | 53.1 | 76.2 | 65.7 | −30.8 | 29.8 | −25.8 | 53.4 |
| D75M | 132.9 | 41.9 | 95.7 | 53.1 | 60.3 | 38.4 | −37.2 | 52.1 | −66.2 | 49.5 |
| D150M | 121.6 | 35.0 | 73.3 | 68.4 | 101.3 | 71.6 | −48.3 | 48.1 | −20.3 | 50.7 |
| P2 | 113.9 | 22.4 | 83.0 | 55.8 | 81.3 | 37.7 | −30.9 | 42.6 | −32.6 | 27.4 |

TABLE 16(b)
WOMAC stiffness index - Percentage change from Baseline

| | 6 MONTHS | | 12 MONTHS | |
|---|---|---|---|---|
| | (VISIT 6) | | (VISIT 7) | |
| TREATMENT GROUPS | Mean | SD | Mean | SD |
| D25M | −44.1 | 33.6 | −61.1 | 28.4 |
| D50M | 38.6 | 153.9 | 52.0 | 157.4 |
| P1 | −36.5 | 27.9 | −30.9 | 45.0 |
| D75M | −24.8 | 44.4 | −49.4 | 30.6 |
| D150M | −46.4 | 46.8 | −23.5 | 47.4 |
| P2 | −31.7 | 41.5 | −30.3 | 26.1 |

### WOMAC Physical Function:

The WOMAC physical function scores across all the treatment groups are comparable at baseline [tables 17(a) and (b)]. The baseline WOMAC physical function scores ranged between about 888.8 in P1 group to about 1094.1 in the D50M group.

WOMAC physical function scores decreased substantially over the study period for all the treatment groups. The maximum mean and percentage change from baseline in the scores across the treatment groups is seen in the D25M group at 6 months (visit 6) and 12 months (visit 7).

In D25M group, the mean (SD) WOMAC physical function score changed from 938.9 (311.1) at baseline to about 528.3 (415.1) at 6 months (visit 6) and about 435.8 (341.3) at 12 months (visit 7). The mean (SD) change from baseline is about 410.6 (454.5) at 6 months (visit 6) (about 43.8%) and about 503.1 (375.1) at 12 months (visit 7) (about 55.6%).

The mean (SD) score in D50M group is about 1094.1 (279.2) at baseline, which changed to about 810.4 (449.6) at 6 months (visit 6) and to about 803.8 (497.1) at 12 months (visit 7). The mean (SD) change from baseline is about 283.7 (486.1) at 6 months (visit 6) (about 22.4%) and about 290.3 (559.2) at 12 months (visit 7) (about 21.6%).

The mean (SD) score in P1 group is about 888.8 (336.1) at baseline, which changed to about 728.0 (442.0) at 6 months (visit 6) and to about 738.2 (530.9) at 12 months (visit 7). The mean (SD) change from baseline is about 160.8 (264.2) at 6 months (visit 6) (20.7%) and 150.6 (457.8) at 12 months (visit 7) (17.9%).

The mean (SD) score in D75M group is 1049.0 (331.9) at baseline, which changed to 698.5 (393.3) at 6 months (visit 6) and to about 605.3 (401.6) at 12 months (visit 7). The mean (SD) change from baseline is about 350.5 (391.2) at 6 months (visit 6) (30.8%) and about 389.7 (538.5) at 12 months (visit 7) (30%).

The mean (SD) score in D150M group is about 983.0 (382.6) at baseline, which changed to 780.5 (574.6) at 6 months (visit 6) and to about 842.9 (511.6) at 12 months (visit 7). The mean (SD) change from baseline is 202.5 (406.9) at 6 months (visit 6) (25.6%) and 140.1 (360.3) at 12 months (visit 7) (17.8%).

The mean (SD) score in P2 group is about 999.9 (246.0) at baseline, which changed to about 712.7 (414.2) at 6 months (visit 6) and to about 765.1 (306.1) at 12 months (visit 7). The mean (SD) change from baseline is about 287.2 (455.9) at 6 months (visit 6) (27.5%) and about 234.8 (347.1) at 12 months (visit 7) (21.4%).

The Wilcoxon signed-rank test (within group analysis) at 6 months (visit 6) (p value=0.0273) and 12 months (visit 7) (p value=0.0039) showed a statistically significant improvement in WOMAC physical function score relative to the baseline for the D25M group while D75M group showed a statistically significant improvement only at 6 months (visit 6) (p value=0.0195) relative to the baseline.

However the ANOVA test showed no statistically significant difference when the mean change from baseline to about 6 months (visit 6) [p value cohort 1: 0.4134, cohort 2: 0.7330] and 12 months (visit 7) [p value cohort 1: 0.2579, cohort 2: 0.4393] is compared within each cohort.

TABLE 17(a)
WOMAC Physical Function - Mean change from Baseline

| | | | | | | | CHANGE FROM | | | |
|---|---|---|---|---|---|---|---|---|---|---|
| | | | POST BASELINE | | | | BASELINE | | | |
| | | | 6 Months | | 12 Months | | 6 Months | | 12 Months | |
| TREATMENT | BASELINE | | (Visit 6) | | (Visit 7) | | (Visit 6) | | (Visit 7) | |
| GROUPS | Mean | SD | Mean | SD | Mean | SD | Mean | SD | Mean | SD |
| D25M | 938.9 | 311.1 | 528.3 | 415.1 | 435.8 | 341.3 | −410.6 | 454.5 | −503.1 | 375.1 |
| D50M | 1094.1 | 279.2 | 810.4 | 449.6 | 803.8 | 497.1 | −283.7 | 486.1 | −290.3 | 559.2 |
| P1 | 888.8 | 336.1 | 728.0 | 442.0 | 738.2 | 530.9 | −160.8 | 264.2 | −150.6 | 457.8 |
| D75M | 1049.0 | 331.9 | 698.5 | 393.3 | 605.3 | 401.6 | −350.5 | 391.2 | −389.7 | 538.5 |
| D150M | 983.0 | 382.6 | 780.5 | 574.6 | 842.9 | 511.6 | −202.5 | 406.9 | −140.1 | 360.3 |
| P2 | 999.9 | 246.0 | 712.7 | 414.2 | 765.1 | 306.1 | −287.2 | 455.9 | −234.8 | 347.1 |

TABLE 17(b)
WOMAC Physical Function - Percentage change from Baseline

| | 6 MONTHS | | 12 MONTHS | |
|---|---|---|---|---|
| | (VISIT 6) | | (VISIT 7) | |
| TREATMENT GROUPS | Mean | SD | Mean | SD |
| D25M | −43.8 | 43.0 | −55.6 | 35.9 |
| D50M | −22.4 | 48.6 | −21.6 | 54.3 |
| P1 | −20.7 | 36.6 | −17.9 | 58.8 |
| D75M | −30.8 | 31.7 | −30.0 | 62.5 |
| D150M | −25.6 | 42.4 | −17.8 | 37.0 |
| P2 | −27.5 | 45.5 | −21.4 | 32.2 |

### WOMAC Composite Index:

The WOMAC composite index scores across all the treatment groups are comparable at baseline [tables 18(a) and (b)]. The baseline WOMAC composite index scores ranged between about 1239.6 in P1 group to about 1498.4 in the D50M group.

WOMAC composite index scores decreased substantially over the study period for all the treatment groups. The maximum mean and percentage change from baseline in the scores across the treatment groups is seen in the D25M group at about 6 months (visit 6) and about 12 months (visit 7).

In D25M group, the mean (SD) WOMAC composite index score changed from about 1315.8 (444.8) at baseline to about 735.7 (591.1) at about 6 months (visit 6) and about 598.0 (461.5) at about 12 months (visit 7). The mean (SD) change from baseline is about 580.1 (612.1) at about 6 months (visit 6) (about 45%) and about 717.8 (503.8) at about 12 months (visit 7) (about 57.1%).

The mean (SD) score in D50M group is about 1498.4 (407.4) at baseline, which changed to 1115.1 (611.9) at 6 months (visit 6) and to 1138.5 (661.1) at 12 months (visit 7). The mean (SD) change from baseline is 383.3 (686.8) at 6 months (visit 6) (20.6%) and 359.9 (786.4) at 12 months (visit 7) (17%).

The mean (SD) score in P1 group is 1239.6 (472.2) at baseline, which changed to 986.7 (602.4) at 6 months (visit 6) and to 1005.8 (750.6) at 12 months (visit 7). The mean (SD) change from baseline is 252.9 (359.8) at 6 months (visit 6) (23.2%) and 233.8 (641.9) at 12 months (visit 7) (20.4%).

The mean (SD) score in D75M group is 1470.6 (471.0) at baseline, which changed to 1016.5 (549.5) at 6 months (visit 6) and to 833.9 (545.8) at 12 months (visit 7). The mean (SD) change from baseline is 454.1 (521.0) at 6 months (visit 6) (28.4%) and 556.0 (707.8) at 12 months (visit 7) (31.9%).

The mean (SD) score in D150M group is 1388.1 (508.8) at baseline, which changed to 1067.3 (803.4) at 6 months (visit 6) and to 1179.4 (720.3) at 12 months (visit 7). The mean (SD) change from baseline is 320.8 (574.8) at 6 months (visit 6) (29.1%) and 208.7 (496.9) at 12 months (visit 7) (19.6%).

The mean (SD) score in P2 group is 1382.0 (324.7) at baseline, which changed to 1000.9 (579.7) at 6 months (visit 6) and to 1075.0 (441.1) at 12 months (visit 7). The mean (SD) change from baseline is 381.1 (588.1) at 6 months (visit 6) (27.8%) and 307.0 (446.8) at 12 months (visit 7) (21.3%).

The Wilcoxon signed-rank test (within group analysis) at 6 months (visit 6) (p value=0.0195) and 12 months (visit 7) (p value=0.0039) showed a statistically significant improvement in WOMAC composite index score relative to the baseline for the D25M groupwhile D75M group showed a statistically significant improvement only at 6 months (visit 6) (p value=0.0137) relative to the baseline.

However the ANOVA test showed no statistically significant difference when the mean change from baseline to 6 months (visit 6) [p value cohort 1: 0.4451, cohort 2: 0.8691] and 12 months (visit 7) [p value cohort 1: 0.2472, cohort 2: 0.3917] is compared within each cohort.

TABLE 18(a)
WOMAC composite index - Mean change from Baseline

| | | | POST BASELINE | | | | CHANGE FROM BASELINE | | | |
|---|---|---|---|---|---|---|---|---|---|---|
| | | | 6 Months | | 12 Months | | 6 Months | | 12 Months | |
| TREATMENT | BASELINE | | (Visit 6) | | (Visit 7) | | (Visit 6) | | (Visit 7) | |
| GROUPS | Mean | SD | Mean | SD | Mean | SD | Mean | SD | Mean | SD |
| D25M | 1315.8 | 444.8 | 735.7 | 591.1 | 598.0 | 461.5 | −580.1 | 612.1 | −717.8 | 503.8 |
| D50M | 1498.4 | 407.4 | 1115.1 | 611.9 | 1138.5 | 661.1 | −383.3 | 686.8 | −359.9 | 786.4 |
| P1 | 1239.6 | 472.2 | 986.7 | 602.4 | 1005.8 | 750.6 | −252.9 | 359.8 | −233.8 | 641.9 |
| D75M | 1470.6 | 471.0 | 1016.5 | 549.5 | 833.9 | 545.8 | −454.1 | 521.0 | −556.0 | 707.8 |
| D150M | 1388.1 | 508.8 | 1067.3 | 803.4 | 1179.4 | 720.3 | −320.8 | 574.8 | −208.7 | 496.9 |
| P2 | 1382.0 | 324.7 | 1000.9 | 579.7 | 1075.0 | 441.1 | −381.1 | 588.1 | −307.0 | 446.8 |

TABLE 18(b)
WOMAC composite index - Percentage change from Baseline

| | 6 MONTHS | | 12 MONTHS | |
|---|---|---|---|---|
| | (VISIT 6) | | (VISIT 7) | |
| TREATMENT GROUPS | Mean | SD | Mean | SD |
| D25M | −45.0 | 41.8 | −57.1 | 33.7 |
| D50M | −20.6 | 52.3 | −17.0 | 57.4 |
| P1 | −23.2 | 36.0 | −20.4 | 58.3 |
| D75M | −28.4 | 31.6 | −31.9 | 59.1 |
| D150M | −29.1 | 44.0 | −19.6 | 37.6 |
| P2 | −27.8 | 43.1 | −21.3 | 30.7 |

Thus overall the D25M group consistently showed statistically significant reduction in mean change from baseline at 6 months (visit 6) and 12 months (visit 7) after treatment when compared to the other dose groups for WOMAC scores.

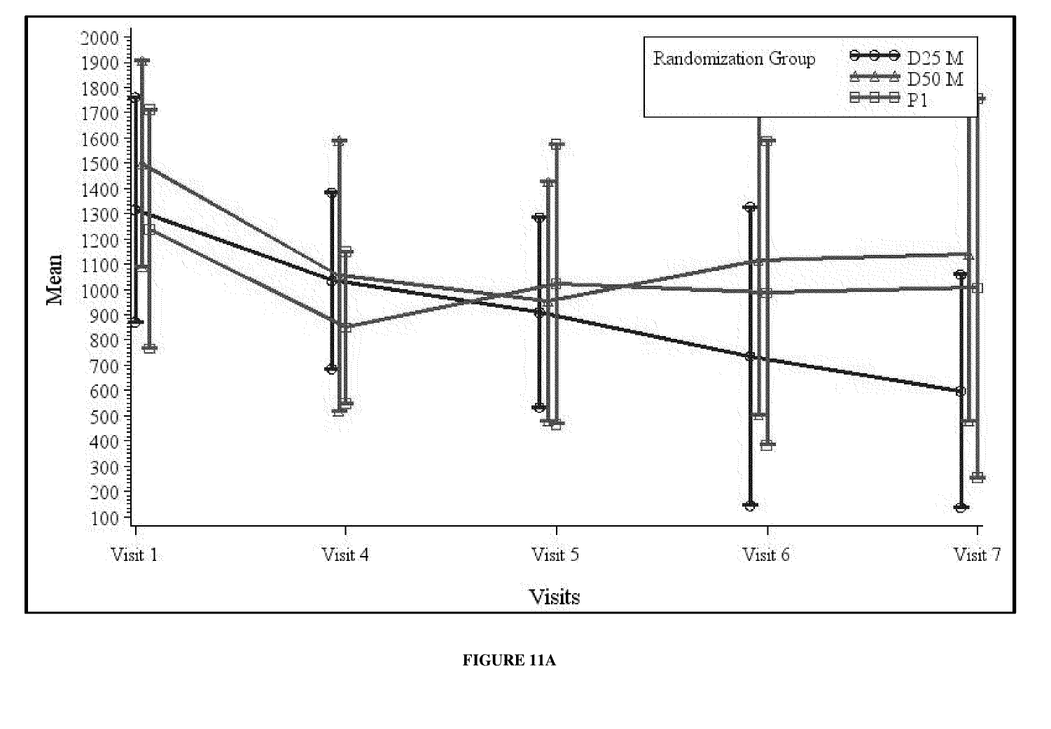

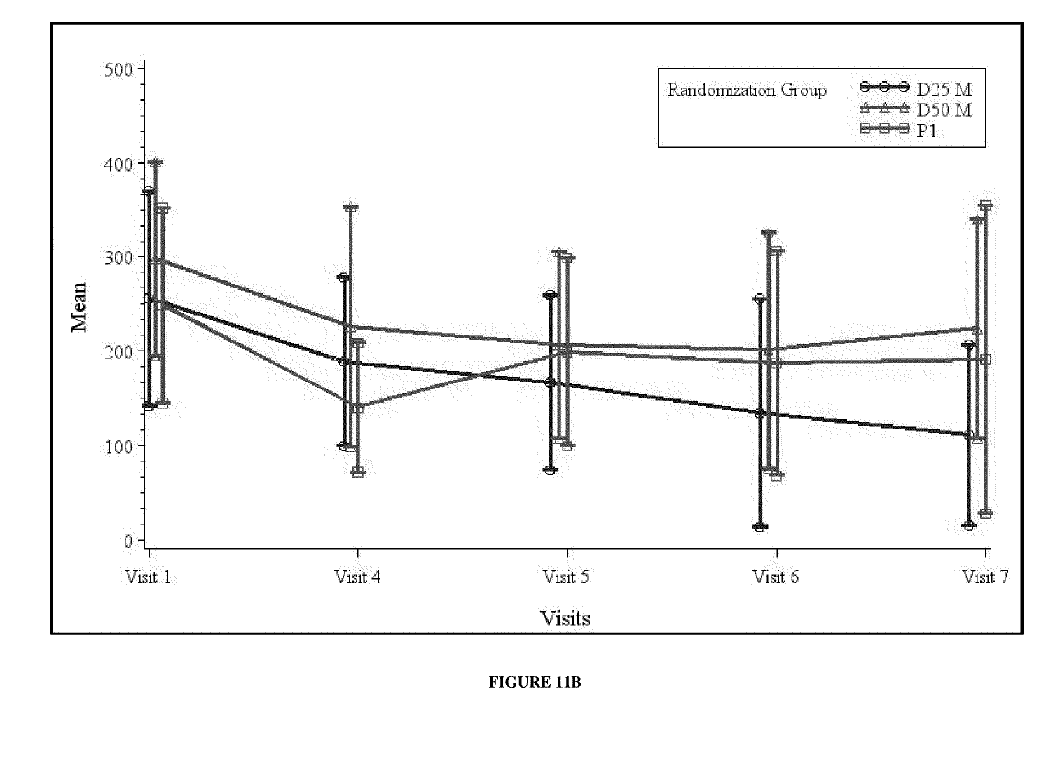

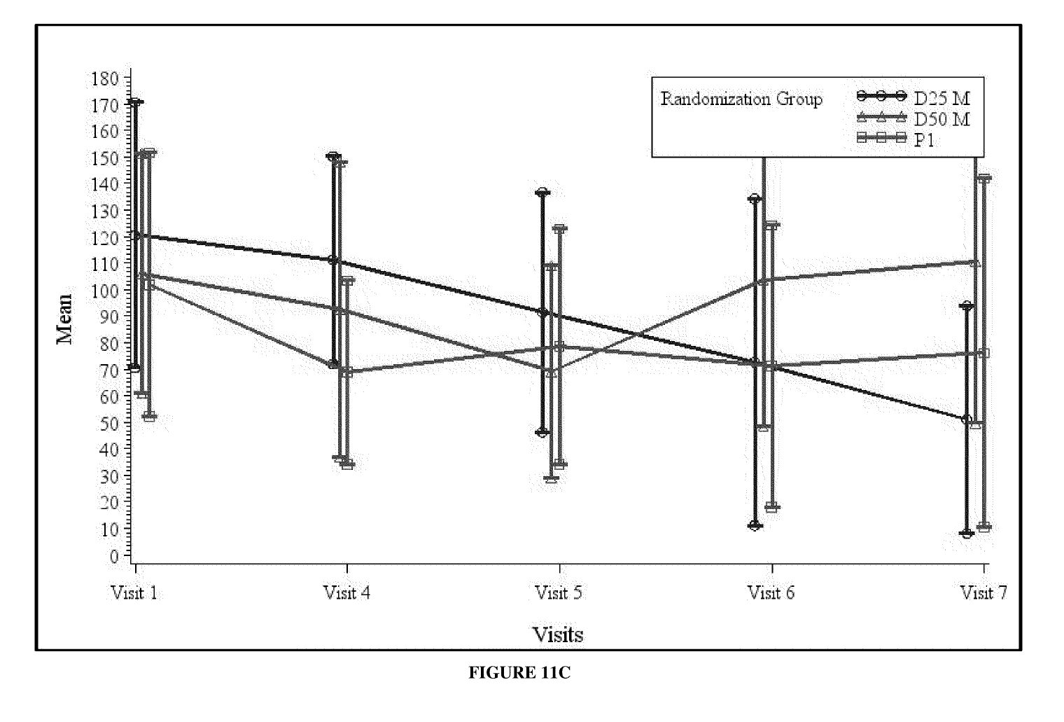

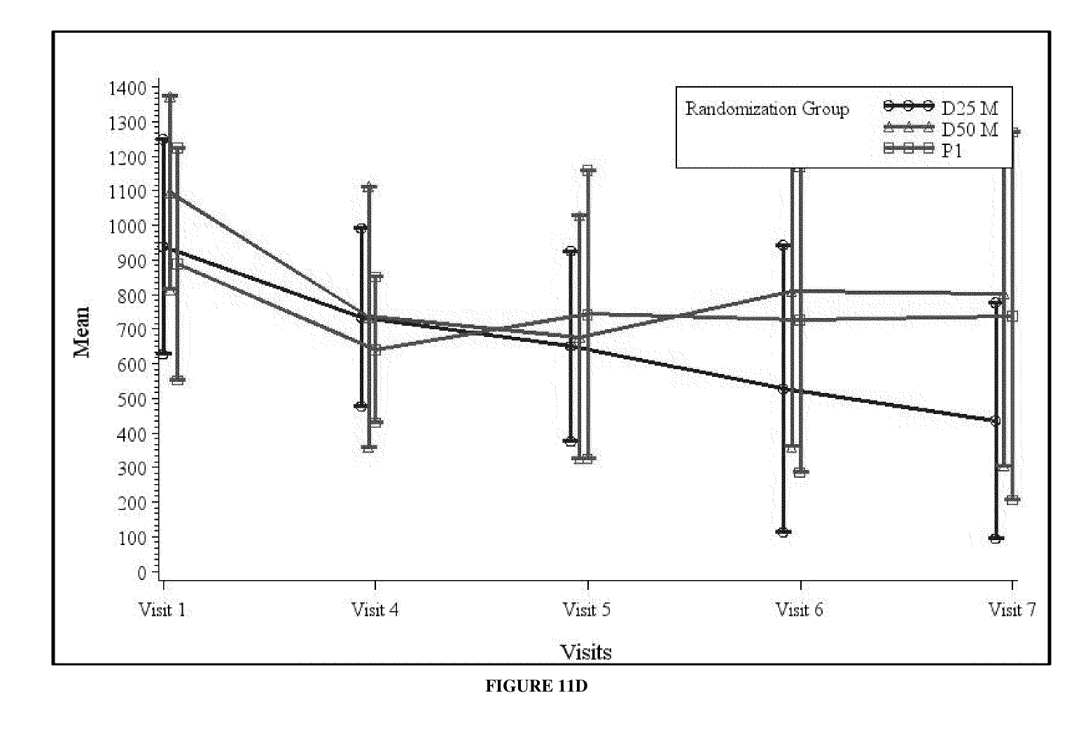

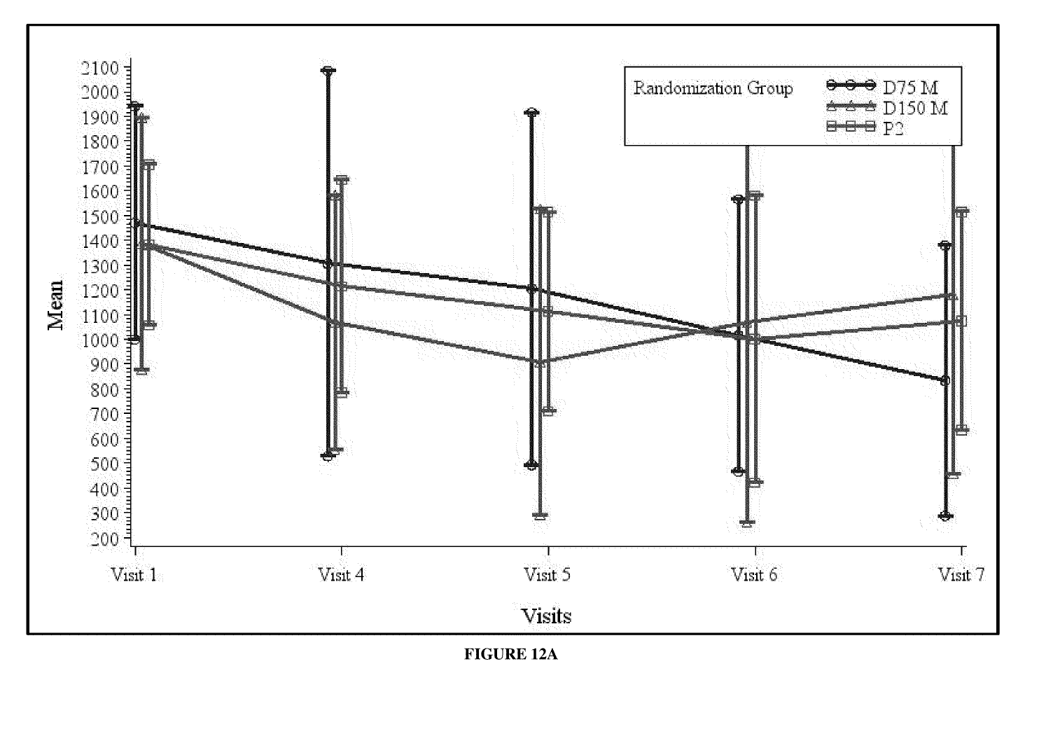

The WOMAC OA index for Cohort 1 i.e. D25M group, D50M group and P1 is determined. The WOMAC total score is improved by about 54% in the 25 Million dose group as compared to about 19% in the placebo arm at one year follow-up. The results are depicted in FIG. 11A.

Similarly the WOMAC Pain is improved by about 56% in the 25 million dose group compared to about 23% in the placebo arm at one year follow-up (FIG. 11B). The WOMAC Stiffness as represented in FIG. 21C is improved by about 58% compared to about 25% in the placebo arm at one year follow-up (FIG. 11C) & the WOMAC Physical Function in the 25 Million dose group as represented in FIG. 21D is improved by about 54% as compared to about 17% in placebo arm. The results are depicted in FIG. 11D

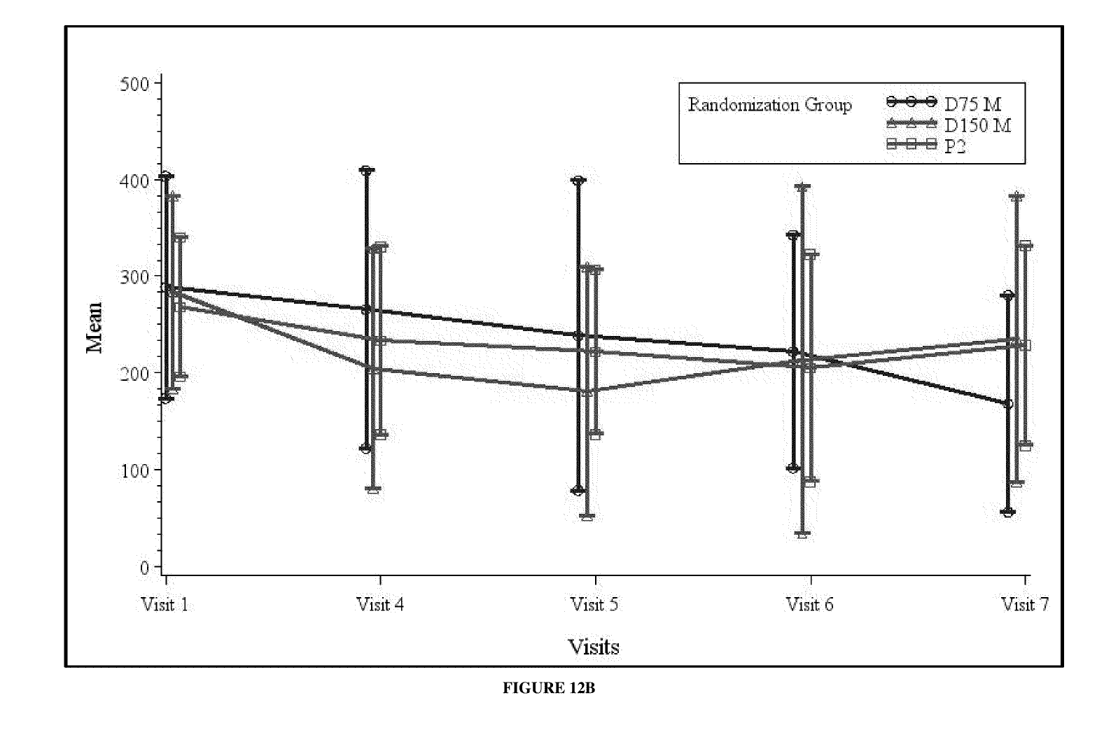

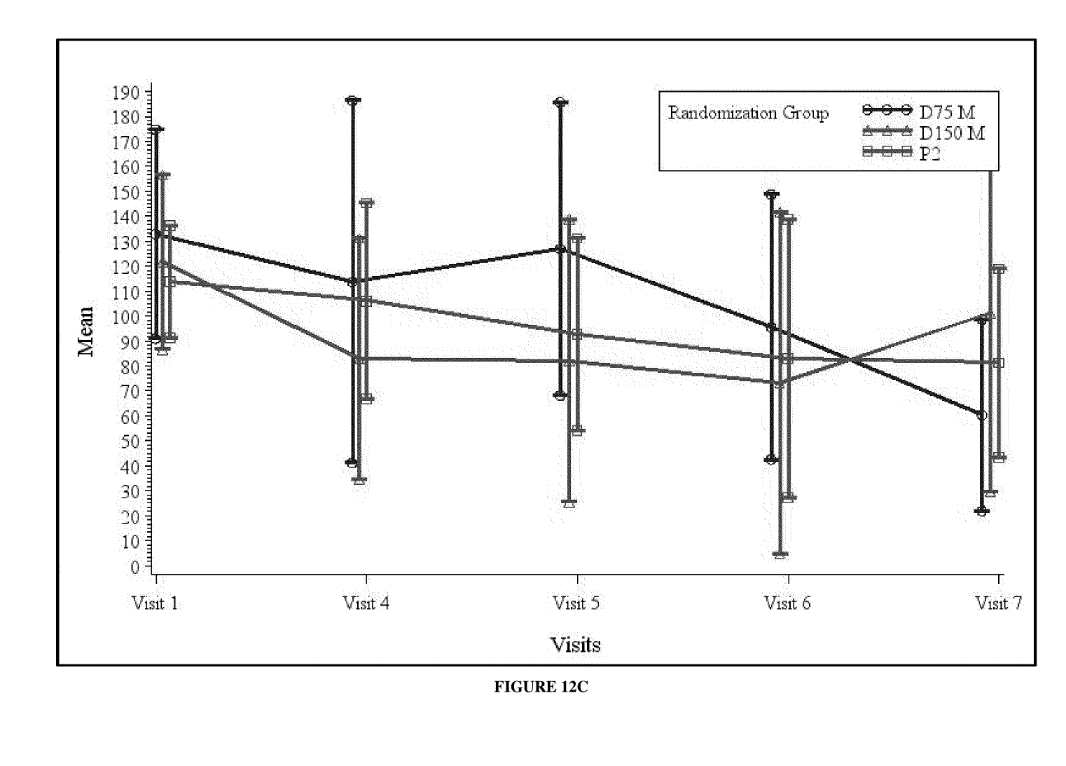

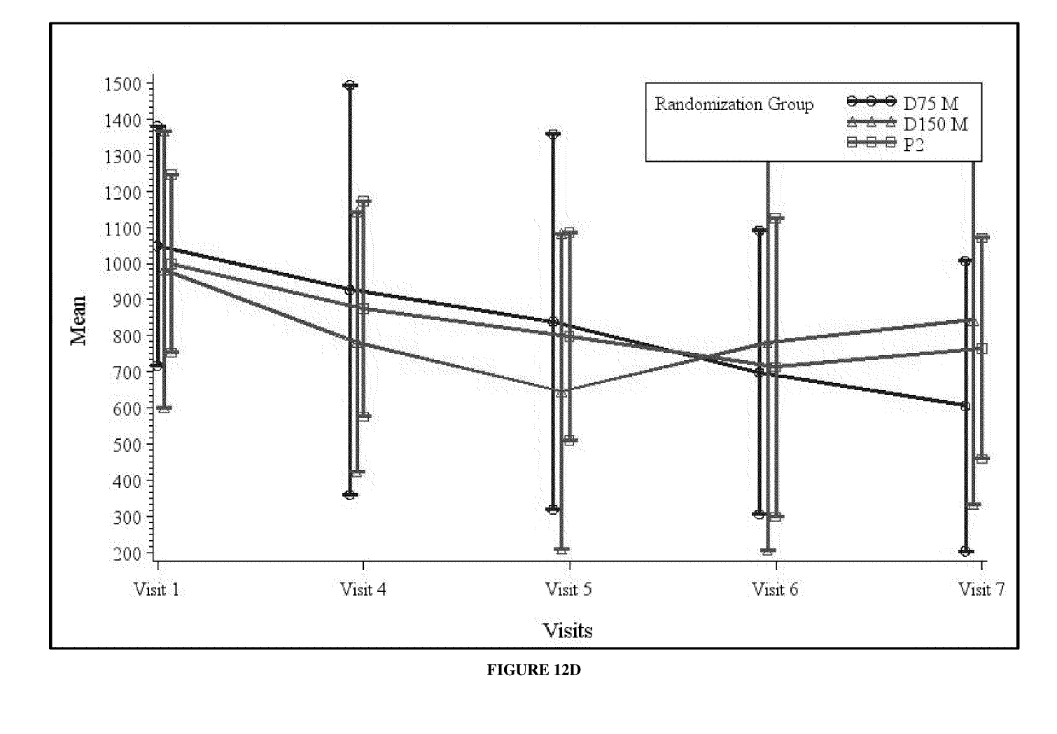

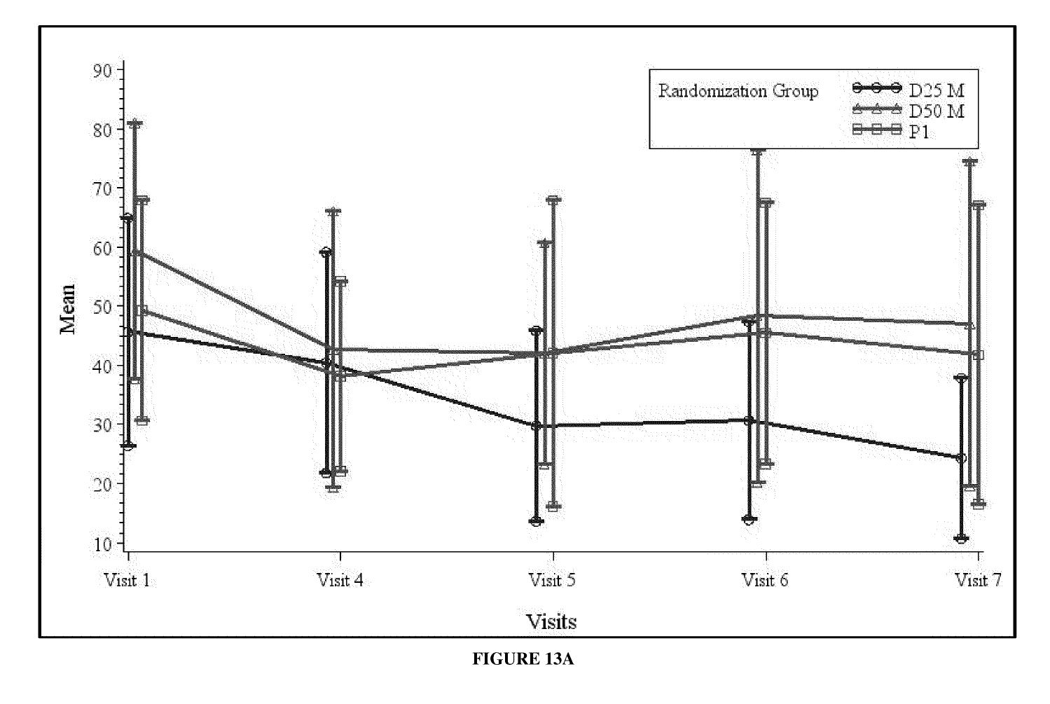

The WOMAC OA index for Cohort 2 i.e. D75M group, D150M group and P2 is determined [ref. FIG. 12A, B, C, D).

The mean (SD) WOMAC Total score or WOMAC composite index score for D75M group, D150M group and P2 is about 1470.6 (471.0), about 1388.1 (508.8) and about 1382.0 (324.7) respectively at baseline visit. The mean (SD) WOMAC composite index score for D75M group, D150M group and P2 at about 1 month (visit 4) is about 1306.6 (778.1), about 1069.7 (513.7) and about 1214.6 (429.7) respectively; at about 3 months (visit 5) is about 1205.2 (711.2), about 908.8 (618.0) and about 1112.4 (401.9) respectively; at about 6 months (visit 6) is about 1016.5 (549.5), about 1067.3 (803.4) and about 1000.9 (579.7) respectively; and at about 12 months (visit 7) is about 833.9 (545.8), about 1179.4 (720.3) and about 1075.0 (441.1) respectively. The results obtained are depicted in FIG. 12A.

The mean (SD) WOMAC pain score for D75M group, D150M group and P2 is about 288.7 (114.9), about 283.5 (99.9) and about 268.2 (72.2) respectively at baseline visit. The mean (SD) WOMAC pain score at subsequent follow up visits for D75M group, D150M group and P2 is about 265.8 (143.6), about 204.5 (123.9) and about 233.4 (97.5) respectively at about 1 month (visit 4); is about 238.9 (160.3), about 181.3 (128.5) and about 221.7 (85.1) respectively at about 3 months (visit 5); is about 222.3 (120.7), about 213.5 (179.5) and about 205.2 (117.5) respectively at about 6 months (visit 6) and is about 168.2 (112.2), about 235.2 (148.1), about 228.6 (103.5) respectively at about 12 months (visit 7). The results obtained are depicted in FIG. 12B.

The mean (SD) WOMAC stiffness score for D75M group, D150M group and P2 is about 132.9 (41.9), about 121.6 (35.0) and about 113.9 (22.4) respectively at baseline visit. The mean (SD) WOMAC stiffness sub score at subsequent follow up visits for D75M group, D150M group and P2 is about 113.8 (72.6), about 82.9 (48.3) and about 106.2 (39.3) respectively at about 1 month (visit 4); is about 127.0 (58.7), about 82.2 (56.7) and about 92.8 (38.5) respectively at about 3 months (visit 5); is about 95.7 (53.1), about 73.3 (68.4) and about 83.0 (55.8) respectively at about 6 months (visit 6); and is about 60.3 (38.4), about 101.3 (71.6) and about 81.3 (37.7) respectively at about 12 months (visit 7). The results obtained are depicted in FIG. 12C.

The mean (SD) WOMAC physical function score for D75M group, D150M group and P2 is about 1049.0 (331.9), about 983.0 (382.6) and about 999.9 (246.0) respectively at baseline visit. The mean (SD) WOMAC physical function score for D75M group, D150M group and P2 at about 1 month (visit 4) is about 927.0 (567.2), about 782.3 (360.9) and about 875.0 (298.8) respectively; at about 3 months (visit 5) is about 839.3 (520.0), about 645.3 (437.5) and about 797.9 (288.2) respectively, at about 6 months (visit 6) is about 698.5 (393.3), about 780.5 (574.6) and about 712.7 (414.2) respectively and at about 12 months (visit 7) is about 605.3 (401.6), about 842.9 (511.6) and about 765.1 (306.1) respectively. The results obtained are depicted in FIG. 12 D.

WOMAC pain index scores decreased substantially over the study period for all the treatment groups. The maximum mean and percentage change from baseline in the scores across the treatment groups is seen in the D25M group at 6 months (visit 6) and 12 months (visit 7).

### Intermittent and Constant Osteoarthritis Pain (ICOAP) Index

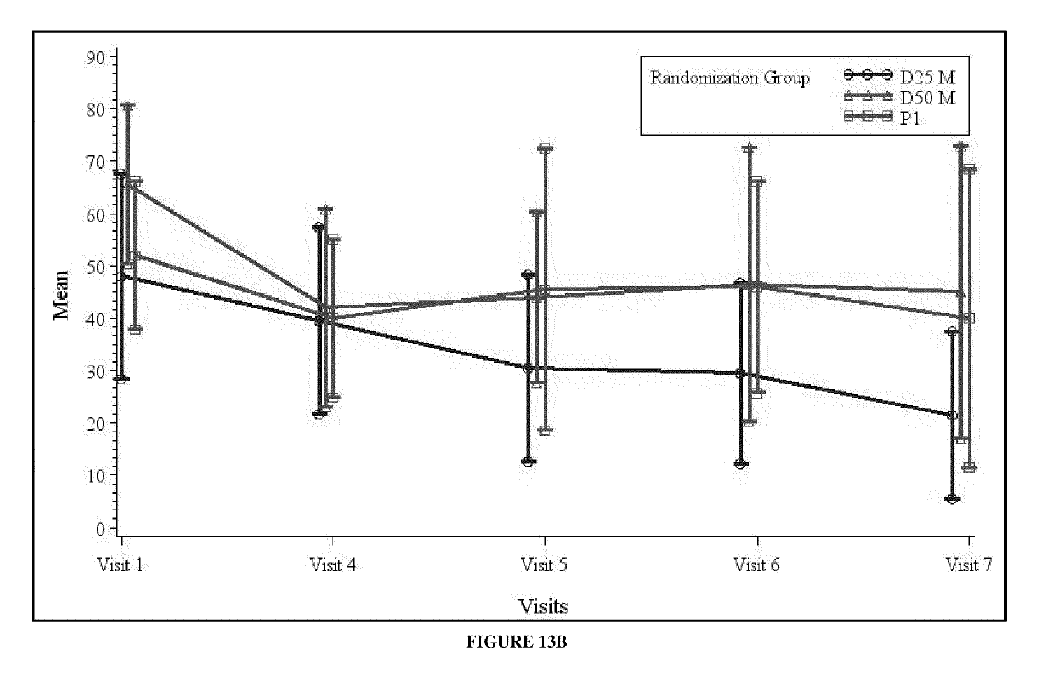

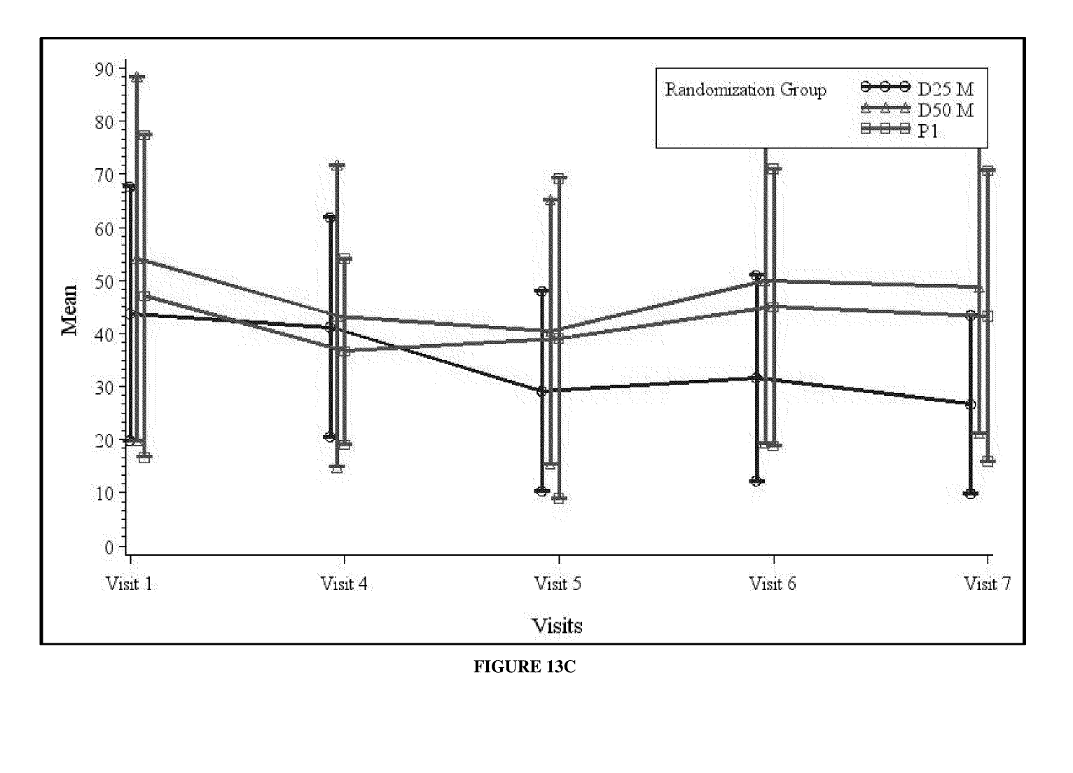

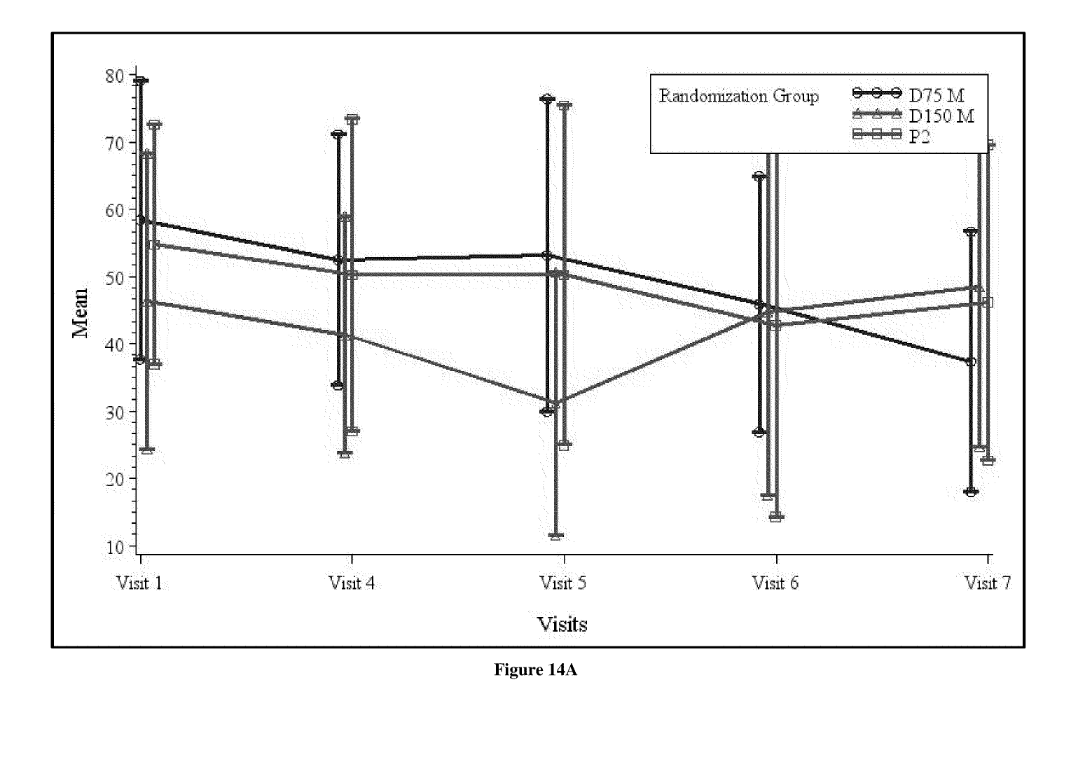

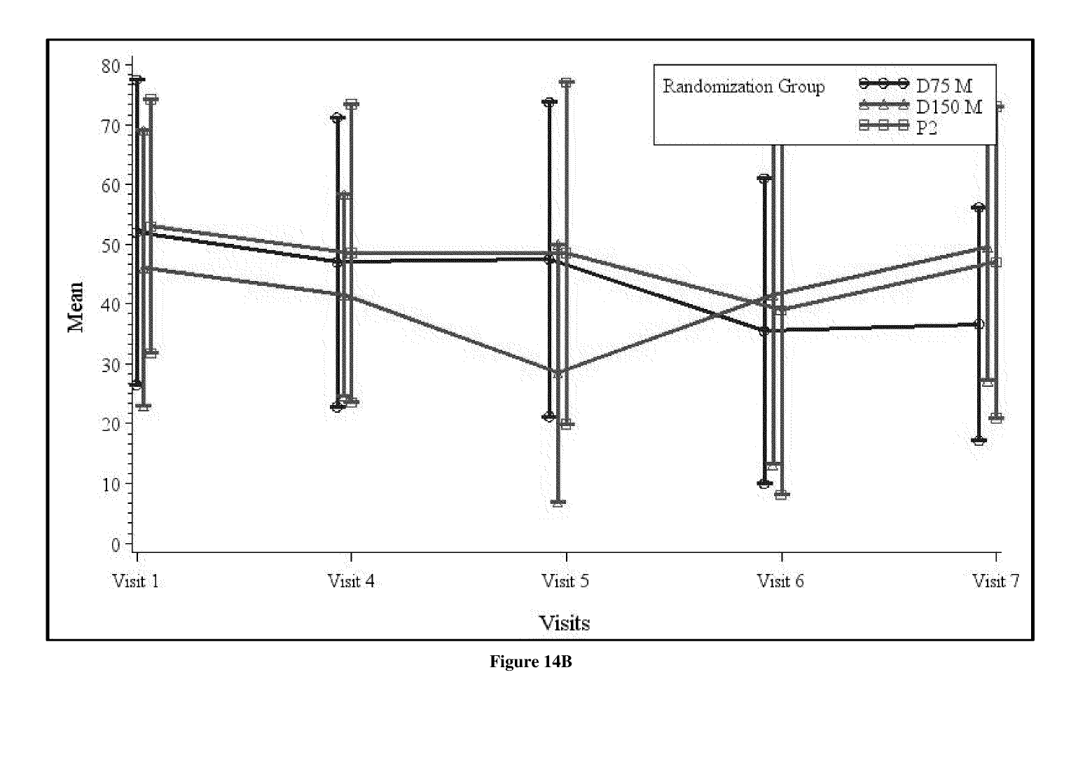

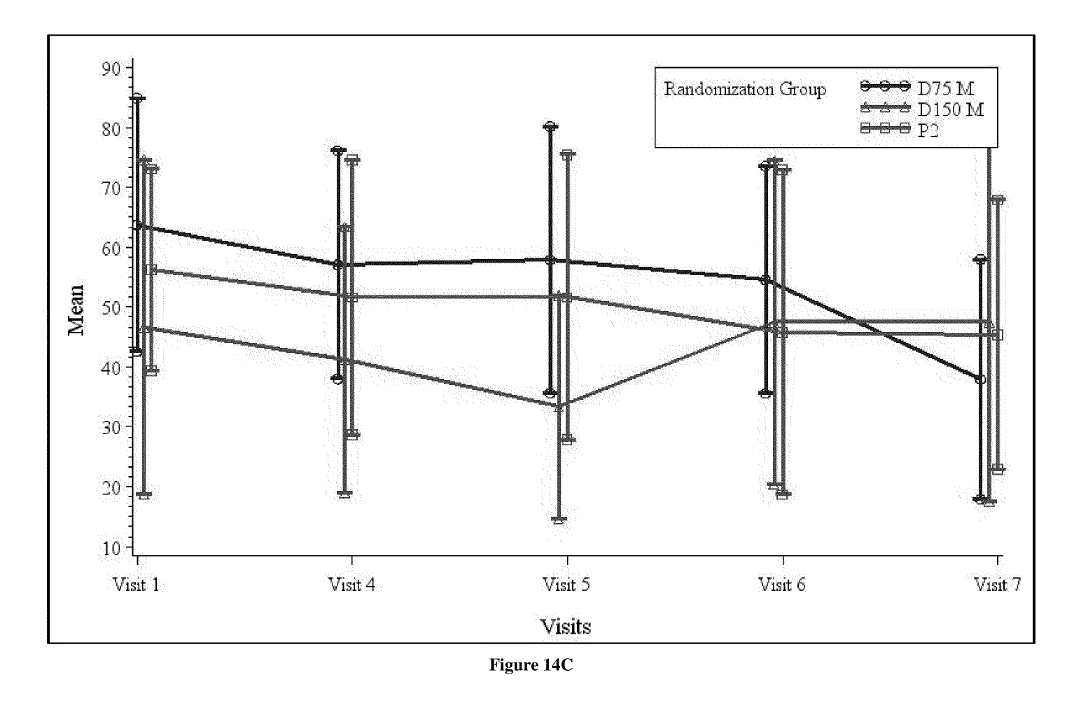

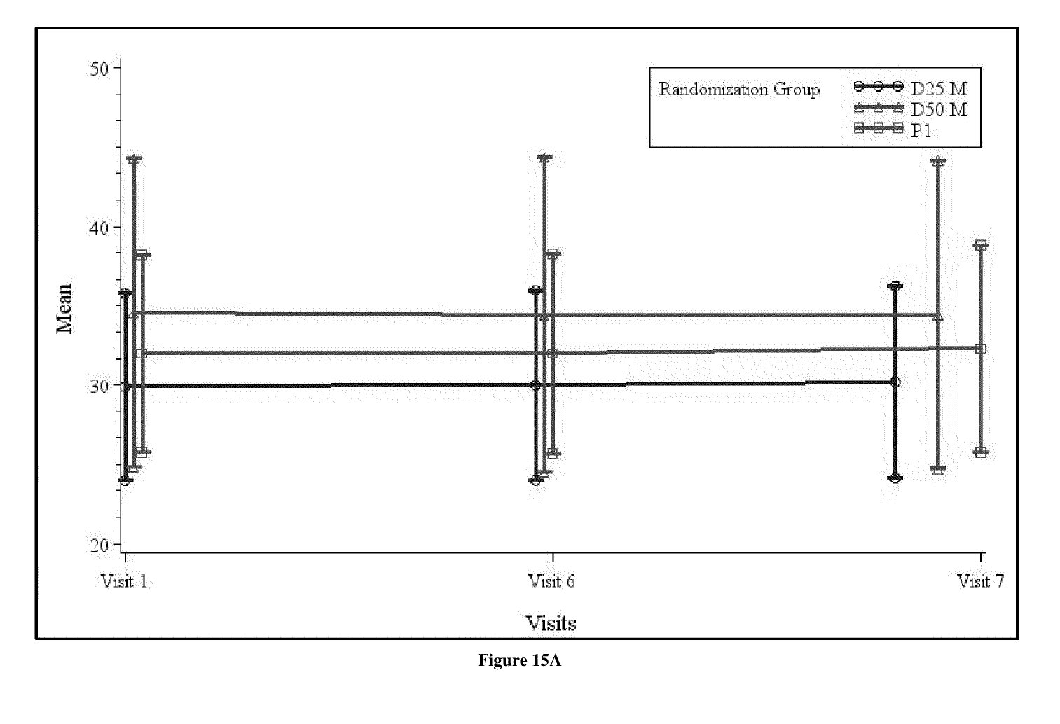

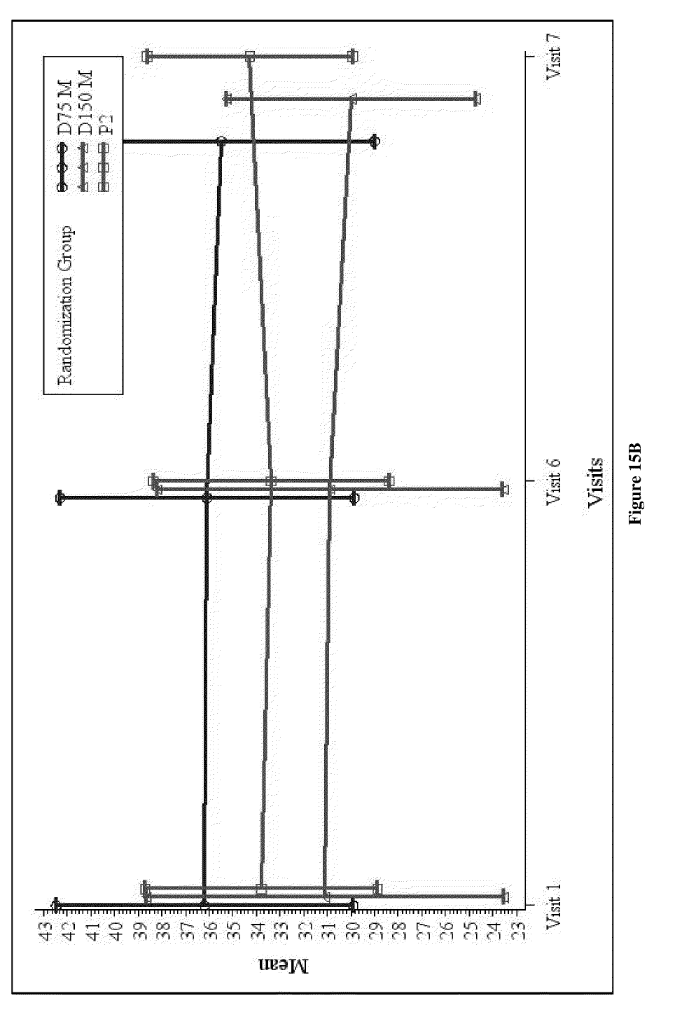

This 11-item tool is designed to assess pain in individuals with hip or knee osteoarthritis taking into account both constant and intermittent pain experiences. The Intermittent and Constant Osteoarthritis Pain (ICOAP) questionnaire is included in the study to assess two forms of pain: intermittent and constant pain. Constant pain is pain that is there all the time, although it may vary in intensity. Constant pain questionnaire includes about five questions which includes pain, aching or discomfort experienced consistently (score range 0-20). Intermittent pain is pain that comes and goes is pain that is not there all the time. Its questionnaire includes six questions which includes knee pain that is brought on predictably by a specific activity or movement (such as walking or stair climbing) but is relieved by rest, as well as pain that may spontaneously occur and then resolve completely (total score 0-24). To calculate the total pain score, sum the constant and intermittent pain subscales. Maximum total pain score ranges from 0-44. Decrease in the score is considered as improvement. The results obtained are depicted in FIGS. 13 A-C for cohort 1 and in FIGS. 14 A-C for cohort 2. Y-axis represents mean ICOAP values and X-axis represents time points of evaluation.

### ICOAP Total Pain Score:

The ICOAP total pain scores across all the treatment groups are comparable at baseline [tables 19(a) and (b)]. The baseline ICOAP total pain scores ranged between 45.7 in D25M group to 59.3 in the D50M group.

ICOAP total pain scores decreased substantially over the study period for all the treatment groups except D150M group where the score at 12 months (Visit 7) is greater than baseline. The maximum mean and percentage change from baseline in the scores across the treatment groups is seen in the D25M group at 6 months (visit 6) and 12 months (visit 7).

In D25M group, the mean (SD) ICOAP total pain score changed from 45.7 (19.2) at baseline to 30.7 (16.7) at 6 months (visit 6) and 24.3 (13.6) at 12 months (visit 7). The mean (SD) change from baseline is 15.0 (26.1) at 6 months (visit 6) (21.7%) and 21.4 (21.2) at 12 months (visit 7) (42.1%).

The mean (SD) score in D50M group is 59.3 (21.7) at baseline, which changed to 48.4 (28.1) at 6 months (visit 6) and to 47.0 (27.4) at 12 months (visit 7). The mean (SD) change from baseline is 10.9 (27.1) at 6 months (visit 6) (14.4%) and 12.3 (27.4) at 12 months (visit 7) (16.7%).

The mean (SD) score in P1 group is 49.3 (18.7) at baseline, which changed to 45.5 (22.1) at 6 months (visit 6) and to 41.8 (25.3) at 12 months (visit 7). The mean (SD) change from baseline is 3.9 (18.3) at 6 months (visit 6) (2.5%) and 7.5 (27.1) at 12 months (visit 7) (10.1%).

The mean (SD) score in D75M group is 58.4 (20.7) at baseline, which changed to 45.9 (19.0) at 6 months (visit 6) and to 37.4 (19.3) at 12 months (visit 7). The mean (SD) change from baseline is 12.5 (9.0) at 6 months (visit 6) (21.1%) and 16.7 (17.6) at 12 months (visit 7) (30.3%).

The mean (SD) score in D150M group is 46.4 (22.0) at baseline, which changed to 44.8 (27.2) at 6 months (visit 6) and to 48.4 (23.7) at 12 months (visit 7). The mean (SD) change from baseline is 1.6 (20.3) at 6 months (visit 6) (increase of 6%) and increase of 2.0 (22.2) at 12 months (visit 7) (increase of 16.8%).

The mean (SD) score in P2 group is 54.8 (17.8) at baseline, which changed to 42.7 (28.5) at 6 months (visit 6) and to 46.1 (23.4) at 12 months (visit 7). The mean (SD) change from baseline is 12.0 (20.7) at 6 months (visit 6) (24.2%) and 8.6 (23.5) at 12 months (visit 7) (15.1%).

The Wilcoxon signed-rank test (within group analysis) at 6 months (visit 6) (p value=0.0020) and 12 months (visit 7) (p value=0.0234) showed a statistically significant improvement in ICOAP total pain score relative to the baseline for the D75M group while D25M group showed a statistically significant improvement only at 6 months (visit 6) (p value=0.0039) relative to the baseline.

However the ANOVA test showed no statistically significant difference when the mean change from baseline to 6 months (visit 6) [p value cohort 1: 0.5866, cohort 2: 0.4732] and 12 months (visit 7) [p value cohort 1: 0.3060, cohort 2: 0.1791] is compared within each cohort.

TABLE 19 (A)
ICOAP total pain - Mean change from baseline

| | | | | | | | CHANGE | | | |
|---|---|---|---|---|---|---|---|---|---|---|
| | | | POST BASELINE | | | | FROM BASELINE | | | |
| | | | 6 Months | | 12 Months | | 6 Months | | 12 Months | |
| TREATMENT | BASELINE | | (Visit 6) | | (Visit 7) | | (Visit 6) | | (Visit 7) | |
| GROUPS | Mean | SD | Mean | SD | Mean | SD | Mean | SD | Mean | SD |
| D25M | 45.7 | 19.2 | 30.7 | 16.7 | 24.3 | 13.6 | −15.0 | 26.1 | −21.4 | 21.2 |
| D50M | 59.3 | 21.7 | 48.4 | 28.1 | 47.0 | 27.4 | −10.9 | 27.1 | −12.3 | 27.4 |
| P1 | 49.3 | 18.7 | 45.5 | 22.1 | 41.8 | 25.3 | −3.9 | 18.3 | −7.5 | 27.1 |
| D75M | 58.4 | 20.7 | 45.9 | 19.0 | 37.4 | 19.3 | −12.5 | 9.0 | −16.7 | 17.6 |
| D150M | 46.4 | 22.0 | 44.8 | 27.2 | 48.4 | 23.7 | −1.6 | 20.3 | 2.0 | 22.2 |
| P2 | 54.8 | 17.8 | 42.7 | 28.5 | 46.1 | 23.4 | −12.0 | 20.7 | −8.6 | 23.5 |

TABLE 19 (b)
ICOAP total pain - Percentage change from baseline

| | 6 MONTHS | | 12 MONTHS | |
|---|---|---|---|---|
| | (VISIT 6) | | (VISIT 7) | |
| TREATMENT GROUPS | Mean | SD | Mean | SD |
| D25M | −21.7 | 59.3 | −42.1 | 36.0 |
| D50M | −14.4 | 53.0 | −16.7 | 55.1 |
| P1 | −2.5 | 49.9 | −10.1 | 49.9 |
| D75M | −21.1 | 13.2 | −30.3 | 39.8 |
| D150M | 6.0 | 64.6 | 16.8 | 48.7 |
| P2 | −24.2 | 40.3 | −15.1 | 42.3 |

### ICOAP Constant Pain Score:

The ICOAP constant pain scores across all the treatment groups are comparable at baseline [tables 20 (a) and (b)]. The baseline ICOAP constant pain scores ranged between 48.0 in D25M group to 65.5 in the D50M group.

ICOAP constant pain scores decreased substantially over the study period for all the treatment groups except D150M group where the score at 12 Months (Visit 7) is greater than baseline. The maximum mean change from baseline in the scores across the treatment groups is seen in the D75M group at 6 months (visit 6) and D25M at 12 months (visit 7). The maximum percentage change from baseline in the scores across the treatment groups is seen in the D50M group at 6 months (visit 6) and D25M at 12 months (visit 7).

In D25M group, the mean (SD) ICOAP constant pain score changed from 48.0 (19.6) at baseline to 29.5 (17.2) at 6 months (visit 6) and 21.5 (16.0) at 12 months (visit 7). The mean (SD) change from baseline is 18.5 (23.8) at 6 months (visit 6) (30.4%) and 26.5 (25.3) at 12 months (visit 7) (48.9%).

The mean (SD) score in D50M group is 65.5 (15.2) at baseline, which changed to 46.5 (26.1) at 6 months (visit 6) and to 45.0 (28.0) at 12 months (visit 7). The mean (SD) change from baseline is 19.0 (27.4) at 6 months (visit 6) (26.8%) and 20.5 (30.2) at 12 months (visit 7) (28.3%).

The mean (SD) score in P1 group is 52.0 (14.2) at baseline, which changed to 46.0 (20.2) at 6 months (visit 6) and to 40.0 (28.5) at 12 months (visit 7). The mean (SD) change from baseline is 6.0 (17.4) at 6 months (visit 6) (11.3%) and 12.0 (31.8) at 12 months (visit 7) (19.2%).

The mean (SD) score in D75M group is 52.0 (25.5) at baseline, which changed to 35.5 (25.5) at 6 months (visit 6) and to 36.7 (19.5) at 12 months (visit 7). The mean (SD) change from baseline is 16.5 (10.0) at 6 months (visit 6) (40.5%) and 10.6 (26.0) at 12 months (visit 7) (increase of 22.6%).

The mean (SD) score in D150M group is 46.0 (23.1) at baseline, which changed to 41.5 (28.3) at 6 months (visit 6) and to 49.5 (22.3) at 12 months (visit 7). The mean (SD) change from baseline is 4.5 (29.6) at 6 months (visit 6) (increase of 38.6%) and increase of 3.5 (21.5) at 12 months (visit 7) (increase of 48.9%).

The mean (SD) score in P2 group is 53.0 (21.2) at baseline, which changed to 39.0 (30.9) at 6 months (visit 6) and to 47.0 (26.1) at 12 months (visit 7). The mean (SD) change from baseline is 14.0 (22.9) at 6 months (visit 6) (32.7%) and 6.0 (22.0) at 12 months (visit 7) (15.3%).

The Wilcoxon signed-rank test (within group analysis) at 6 months (visit 6) (p value=0.0352) and 12 months (visit 7) (p value=0.0078) showed a statistically significant improvement in ICOAP constant pain score relative to the baseline for the D25M group while D75M group showed a statistically significant improvement only at 6 months (visit 6) (p value=0.0039) relative to the baseline.

However the ANOVA test showed no statistically significant difference when the mean change from baseline to 6 months (visit 6) [p value cohort 1: 0.3795, cohort 2: 0.4594] and 12 months (visit 7) [p value cohort 1: 0.5450, cohort 2: 0.4097] is compared within each cohort.

TABLE 20(a)
ICOAP constant pain - Mean change from Baseline

| | | | | | | | CHANGE | | | |
|---|---|---|---|---|---|---|---|---|---|---|
| | | | POST BASELINE | | | | FROM BASELINE | | | |
| | | | 6 Months | | 12 Months | | 6 Months | | 12 Months | |
| TREATMENT | BASELINE | | (Visit 6) | | (Visit 7) | | (Visit 6) | | (Visit 7) | |
| GROUPS | Mean | SD | Mean | SD | Mean | SD | Mean | SD | Mean | SD |
| D25M | 48.0 | 19.6 | 29.5 | 17.2 | 21.5 | 16.0 | −18.5 | 23.8 | −26.5 | 25.3 |
| D50M | 65.5 | 15.2 | 46.5 | 26.1 | 45.0 | 28.0 | −19.0 | 27.4 | −20.5 | 30.2 |
| P1 | 52.0 | 14.2 | 46.0 | 20.2 | 40.0 | 28.5 | −6.0 | 17.4 | −12.0 | 31.8 |
| D75M | 52.0 | 25.5 | 35.5 | 25.5 | 36.7 | 19.5 | −16.5 | 10.0 | −10.6 | 26.0 |
| D150M | 46.0 | 23.1 | 41.5 | 28.3 | 49.5 | 22.3 | −4.5 | 29.6 | 3.5 | 21.5 |
| P2 | 53.0 | 21.2 | 39.0 | 30.9 | 47.0 | 26.1 | −14.0 | 22.9 | −6.0 | 22.0 |

TABLE 20(b)
ICOAP constant pain - Percentage change from Baseline
ICOAP constant pain - Percentage change from Baseline

| | 6 Months (Visit 6) | | 12 Months (Visit 7) | |
|---|---|---|---|---|
| Treatment groups | Mean | SD | Mean | SD |
| D25M | −30.4 | 57.0 | −48.9 | 41.4 |
| D50M | −26.8 | 41.8 | −28.3 | 45.7 |
| P1 | −11.3 | 33.5 | −19.2 | 54.7 |
| D75M | −40.5 | 34.7 | 22.6 | 182.1 |
| D150M | 38.6 | 170.2 | 48.9 | 129.2 |
| P2 | −32.7 | 47.0 | −15.3 | 47.1 |

### ICOAP Intermittent Pain Score:

The ICOAP intermittent pain scores across all the treatment groups are comparable at baseline [tables 21(a) and (b)]. The baseline ICOAP intermittent pain scores ranged between 43.8 in D25M group to 63.7 in the D75M group.

ICOAP intermittent pain scores decreased substantially over the study period for all the treatment groups except D150M group where the score at 12 Months (Visit 7) is greater than baseline. The maximum mean change from baseline in the scores across the treatment groups is seen in the D25M group at 6 months (visit 6) and in D75M at 12 months (visit 7). The maximum percentage change from baseline in the scores across the treatment groups is seen in the D25M group at 6 months (visit 6) and 12 months (visit 7).

In D25M group, the mean (SD) ICOAP intermittent pain score changed from 43.8 (23.9) at baseline to 31.7 (19.4) at 6 months (visit 6) and 26.7 (16.8) at 12 months (visit 7). The mean (SD) change from baseline is 12.1 (32.4) at 6 months (visit 6) (25.8%) and 17.1 (28.4) at 12 months (visit 7) (42.6%).

The mean (SD) score in D50M group is 54.2 (34.2) at baseline, which changed to 50.0 (30.7) at 6 months (visit 6) and to 48.8 (27.6) at 12 months (visit 7). The mean (SD) change from baseline is 4.2 (33.0) at 6 months (visit 6) (15.8%) and 5.4 (33.1) at 12 months (visit 7) (20.2%).

The mean (SD) score in P1 group is 47.1 (30.4) at baseline, which changed to 45.0 (26.1) at 6 months (visit 6) and to 43.3 (27.4) at 12 months (visit 7). The mean (SD) change from baseline is 2.1 (25.5) at 6 months (visit 6) (11.3%) and 3.8 (26.3) at 12 months (visit 7) (19.2%).

The mean (SD) score in D75M group is 63.7 (21.2) at baseline, which changed to 54.6 (19.0) at 6 months (visit 6) and to 38.0 (20.0) at 12 months (visit 7). The mean (SD) change from baseline is 9.2 (14.4) at 6 months (visit 6) (9.6%) and 21.8 (14.2) at 12 months (visit 7) (37.2%).

The mean (SD) score in D150M group is 46.7 (28.0) at baseline, which changed to 47.5 (27.0) at 6 months (visit 6) and to 47.5 (29.9) at 12 months (visit 7). The mean (SD) change from baseline is an increase of 0.8 (21.3) at 6 months (visit 6) (increase of 1.9%) and increase of 0.8 (24.5) at 12 months (visit 7) (increase of 12.4%).

The mean (SD) score in P2 group is 56.3 (16.8) at baseline, which changed to 45.8 (27.1) at 6 months (visit 6) and to 45.4 (22.5) at 12 months (visit 7). The mean (SD) change from baseline is 10.4 (19.3) at 6 months (visit 6) (19.8%) and 10.8 (25.9) at 12 months (visit 7) (16.3%).

There is no statistically significant difference observed when the mean change is compared relative to the baseline at 6 months (visit 6) and at 12 months (visit 7) using Wilcoxon signed-rank test (within group analysis).

There is no statistically significant difference observed when the mean change from baseline to 6 months (visit 6) and 12 months (visit 7) is compared within each cohort using ANOVA test.

However a statistically significant difference (p value=0.0272) is observed when the mean differences of D75M and D150M groups are compared (using t-test). The D75M group showed a higher mean change compared to D150M group (D75M vs. D150M: −21.8 vs. 0.8).

TABLE 21(a)
ICOAP intermittent pain - Mean change from Baseline
ICOAP intermittent pain - Mean change from Baseline

| | | | Post Baseline | | | | Change from Baseline | | | |
|---|---|---|---|---|---|---|---|---|---|---|
| | | | 6 Months | | 12 Months | | 6 Months | | 12 Months | |
| Treatment | Baseline | | (Visit 6) | | (Visit 7) | | (Visit 6) | | (Visit 7) | |
| groups | Mean | SD | Mean | SD | Mean | SD | Mean | SD | Mean | SD |
| D25M | 43.8 | 23.9 | 31.7 | 19.4 | 26.7 | 16.8 | −12.1 | 32.4 | −17.1 | 28.4 |
| D50M | 54.2 | 34.2 | 50.0 | 30.7 | 48.8 | 27.6 | −4.2 | 33.0 | −5.4 | 33.1 |
| P1 | 47.1 | 30.4 | 45.0 | 26.1 | 43.3 | 27.4 | −2.1 | 25.5 | −3.8 | 26.3 |
| D75M | 63.7 | 21.2 | 54.6 | 19.0 | 38.0 | 20.0 | −9.2 | 14.4 | −21.8 | 14.2 |
| D150M | 46.7 | 28.0 | 47.5 | 27.0 | 47.5 | 29.9 | 0.8 | 21.3 | 0.8 | 24.5 |
| P2 | 56.3 | 16.8 | 45.8 | 27.1 | 45.4 | 22.5 | −10.4 | 19.3 | −10.8 | 25.9 |

TABLE 21(b)
ICOAP intermittent pain - Percentage change from Baseline
ICOAP intermittent pain - Percentage change from Baseline

| | 6 Months (Visit 6) | | 12 Months (Visit 7) | |
|---|---|---|---|---|
| Treatment groups | Mean | SD | Mean | SD |
| D25M | −25.8 | 62.8 | −42.6 | 41.4 |
| D50M | −15.8 | 36.5 | −20.2 | 32.9 |
| P1 | −12.6 | 40.4 | −9.2 | 49.0 |
| D75M | −9.6 | 31.1 | −37.2 | 30.0 |
| D150M | 1.9 | 50.4 | 12.4 | 48.6 |
| P2 | −19.8 | 38.7 | −16.3 | 45.0 |

### Cohort 1

### A: ICOAP Total Pain

The mean (SD) ICOAP Total score for D25M group, D50M group and P1 is about 45.7 (19.2), about 59.3 (21.7) and about 49.3 (18.7) respectively at baseline visit. The mean (SD) ICOAP Total score for D25M group, D50M group and P1 is about 40.5 (18.7), about 42.7 (23.4) and about 38.2 (16.1) respectively at about 1 month (visit 4); is about 29.8 (16.1), about 42.0 (18.7) and about 42.0 (25.9) respectively at about 3 months (visit 5); is about 30.7 (16.7), about 48.4 (28.1) and about 45.5 (22.1) respectively at about 6 months (visit 6); and is about 24.3 (13.6), about 47.0 (27.4) and about 41.1 (25.3) respectively at about 12 months (visit 7). Among cohort 1 (25M, 50M, P1), 25M dose group showed consistent improvement in ICOAP values, which is evident from about 3 months (visit 5) till about 12 months (visit 7). The results obtained are depicted in FIG. 13 A.

### B: ICOAP Constant Pain

The mean (SD) ICOAP Constant score for D25M group, D50M group and P1 is about 48.0 (19.6), about 65.5 (15.2) and about 52.0 (14.2) respectively at baseline. The mean (SD) ICOAP Constant score for D25M group, D50M group and P1 is about 39.5 (17.9), about 42.0 (18.9) and about 40.0 (15.1) respectively at about 1 month (visit 4; is about 29.8 (16.1), about 42.0 (18.7) and about 42.0 (25.9) respectively at about 3 months (visit 5); is about 30.7 (16.7), about 48.4 (28.1) and about 45.5 (22.1) respectively at about 6 months (visit 6); and is about 24.3 (13.6), about 47.0 (27.4) and about 41.1 (25.3) respectively at about 12 months (visit 7). Among cohort 1 (25M, 50M, P1), 25M dose group showed consistent improvement in ICOAP values, which is evident from about 3 months (visit 5) till about 12 months (visit 7). The results obtained are depicted in FIG. 13 B.

### C: ICOAP Intermittent Pain

The mean (SD) ICOAP Constant score for D25M group, D50M group and P1 is about 43.8 (23.9), about 54.2 (34.2), about 47.1 (30.4) respectively at baseline. The mean (SD) ICOAP Constant score for D25M group, D50M group and P1 is about 41.3 (20.6), about 43.3 (28.5), about 36.7 (17.5) respectively at about 1 month (visit 4); is about 29.2 (18.8), about 40.4 (24.9), about 39.2 (30.1) respectively at about 3 months (visit 5); is about 31.7 (19.4), about 50.0 (30.7), about 45.0 (about 26.1) respectively at about 6 months (visit 6); and is about 26.7 (16.8), about 48.8 (27.6), about 43.3 (27.4) respectively at about 12 months (visit 7). Among cohort 1 (25M, 50M, P1), 25M dose group showed consistent improvement in ICOAP values, which is evident from 3 months (visit 5) till 12 months (visit 7). The results obtained are depicted in FIG. 13 C.

ICOAP total scores reduce by about 47% in the 25 Million dose group as compared to about 17% in the cell arm after one year follow-up. There is similar improvement in the ICOAP Constant pain and ICOAP intermittent pain in the 25 Million dose group (FIGS. 23 A-C). There is also a marginal increase in cartilage thickness seen at six months interval in the lower doses.

### Cohort 2

### A: ICOAP Total Pain

The mean (SD) ICOAP Total score for D75M group, D150M group and P2 is about 58.4 (20.7), about 46.4 (22.0) and about 54.8 (17.8) respectively at baseline visit. The mean (SD) ICOAP Total score for D75M group, D150M group and P2 is about 52.5 (18.7), about 41.4 (17.6) and about 50.2 (23.2) respectively at 1 month (visit 4); is about 53.2 (23.2), about 31.1 (19.6) and about 50.2 (25.2) respectively at about 3 months (visit 5); is about 45.9 (19.0), about 44.8 (27.2) and about 42.7 (28.5) respectively at about 6 months (visit 6); and is about 37.4 (19.3), about 48.4 (23.7) and about 46.1 (23.4) respectively at about 12 months (visit 7). The results obtained are depicted in FIG. 14 A.

### B: ICOAP Constant Pain

The mean (SD) ICOAP Total score for D75M group, D150M group and P2 is about 52.0 (25.5), about 46.0 (23.1), about 53.0 (21.2) respectively at baseline visit. The mean (SD) ICOAP Total score for D75M group, D150M group and P2 is about 47.0 (24.2), about 41.5 (16.8), about 48.5 (24.9) respectively at about 1 month (visit 4); is about 47.5 (26.3), about 28.5 (21.5), about 48.5 (28.6) respectively at about 3 months (visit 5); is about 35.5 (25.5), about 41.5 (28.3), about 39.0 (30.9) respectively at about 6 months (visit 6); and is about 36.7 (19.5), about 49.5 (22.3), about 47.0 (26.1) respectively at about 12 months (visit 7). The results obtained are depicted in FIG. 14B.

### C: ICOAP Intermittent Pain

The mean (SD) ICOAP Total score for D75M group, D150M group and P2 is about 63.7 (21.2), about 46.7 (28.0), about 56.3 (16.8) respectively at baseline visit. The mean (SD) ICOAP Total score for D75M group, D150M group and P2 is about 57.1 (19.0), about 41.2 (22.2), about 51.7 (23.0) respectively at about 1 month (visit 4); is about 57.9 (22.3), about 33.3 (18.6), about 51.7 (23.8) respectively at about 3 months (visit 5); is about 54.6 (19.0), about 47.5 (27.0), about 45.8 (27.1) respectively at about 6 months (visit 6); and is about 38.0 (20.0), about 47.5 (29.9), about 45.4 (22.5) respectively at about 12 months (visit 7). The results obtained are depicted in FIG. 14C.

### Assessing Cartilage Signal and Morphology Using Whole Organ Magnetic Resonance Imaging Score (WORMS) and Assessment of Cartilage Thickness Using MRI

Magnetic resonance Imaging (MRI) of knee(s) of patients under Cohorts 1 and 2 is done as per standard techniques for assessing cartilage thickness, using MRI at different points of the knee joint and to determine Whole Organ Magnetic Resonance Imaging Score (WORM) score.

### Cartilage Thickness:

The cartilage thickness is scored at multiple compartment of the knee joint. Multiple points are assessed for the cartilage thickness at patello femoral joint, lateral femoral tibial joint and the medial tibial femoral joint. The Thickness of the cartilage is assessed in millimeters and the totaled for the individual compartment. The compartment scores are added to obtain the total knee joint score.

### Scoring for Cartilage Thickness:

A total of 14 measurements of cartilage thickness is recorded at each examination/on each patient visit using the sequence. The sum of these measurements represents the cartilage score for that visit. This score is used as the metric to track cartilage regeneration.

In summary, the safety results demonstrate that ex-vivo cultured adult allogeneic MSCs have a favorable safety profile when injected intra-articularly for osteoarthritis of knee. Complications related to the treatment like injection site reactions and hypersensitivity to study drug is more prevalent in the D75M dose group and D150M dose group when compared to that of D25M and D50M dose group.

The results of the study shows that the improvement in subjective parameters are seen in a pronounced manner in 25 million dose group when compared to other groups within the same cohort.

In conclusion, this randomized, controlled clinical trial demonstrated that a single intra-articular injection of ex-vivo cultured adult allogeneic MSCs is a safe and effective treatment for osteoarthritis. The intra-articular injection of allogeneic mesenchymal stromal cells at a dose of about 25 million cells have shown efficacy trend with acceptable safety profile in osteoarthritis of knee.

### CONCLUSION

Pooled allogeneic BM-MSCs injected intra-articularly at a number of about 10-60 million cells, preferably about 25 million cells in the injectable solution/the cell composition, show improved efficacy in treating OA by improvement in pain and cartilage regeneration. In particular, efficacy parameters such as PAM score, adverse events, VAS score, WOMAC index, ICOAP index and WORMS show significant improvement when compared to placebo.

Thus, the present disclosure is able to successfully overcome the various deficiencies of prior art and provides for bone marrow derived pooled allogenic MSC compositions with specific dosage ranges and modes of administration.

Additional embodiments and features of the present disclosure will be apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein.

The foregoing description of the specific embodiments fully reveals the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

Throughout this specification, the word “comprise”, or variations such as “comprises” or “comprising” wherever used, will be understood to imply the inclusion of a stated element, integer or step, or group of elements, integers or steps, but not the exclusion of any other element, integer or step, or group of elements, integers or steps.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles and the like that has been included in this specification is solely for the purpose of providing a context for the disclosure. It is not to be taken as an admission that any or all of these matters form a part of the prior art base or are common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1-15. (canceled)

16. A composition for treating Osteoarthritis comprising pooled allogeneic bone marrow derived mesenchymal stromal cells ranging from about 10 million cells to about 60 million cells, optionally along with carrier, cyropreservant or protein, or any combinations thereof.

17. The composition as claimed in claim 16, wherein the mesenchymal stromal cells range from about 20 million cells to about 50 million cells, more preferably at about 25 million cells.

18. The composition as claimed in claim 16, wherein the pooled allogeneic mesenchymal stromal cells are obtained by combining mesenchymal stromal cells from at least two donors, preferably about 2 donors to about 10 donors, more preferably about three donors.

19. The composition as claimed in claim 16, wherein the pooled allogeneic mesenchymal stromal cells have plurality of HLA genotypes at loci selected from group comprising HLA-A, HLA-B, HLA-C, HLA-DQ and HLA-DR or any combinations thereof; wherein the pooled mesenchymal stromal cells have sGAG (sulfated glycosaminoglycan) value ranging from about 25 μg to about 50 μg; and wherein at least 80% of the mesenchymal stromal cells are positive for cell specific markers selected from group comprising CD 73, CD 105, CD 44, CD90 and CD166 cells or any combinations thereof; and at least 90% of the mesenchymal stromal cells are negative for markers selected from group comprising CD34, CD45, CD133, CD14, CD19 and HLA-DR or any combinations thereof.

20. The composition as claimed in claim 16, wherein the carrier is multiple electrolyte solution at a concentration ranging from about 80% to about 90%; the cyropreservant is Dimethyl Sulfoxide (DMSO) at concentration ranging from about 2% to about 10%, or cryopreservation medium comprising DMSO at concentration ranging from about 2% to about 10%; the protein is human serum albumin (HSA) at a concentration ranging from about 1% to about 6%.

21. The composition as claimed in claim 16, wherein the composition is formulated as a formulation selected from group comprising aqueous suspension, emulsion, drop, emulsion in hard or soft gel capsule, elixir, lyophilized cell powder and cell spray or any combinations thereof, preferably aqueous suspension.

22. The composition as claimed in claim 16, wherein the composition is administered as single dose or multiple doses, preferably as single dose, at one or multiple sites through mode selected from group comprising intraarticular administration, intramuscular administration, intravenous administration or any combinations thereof, preferably intraarticular administration.

23. A method of treating Osteoarthritis, said method comprising acts of administering a composition comprising pooled allogeneic bone marrow derived mesenchymal stromal cells ranging from about 10 million cells to about 60 million cells, optionally along with carrier, cyropreservant or protein, or any combinations thereof, and optionally administering hyaluronic acid to subject in need thereof for treating the Osteoarthritis.

24. The method as claimed in claim 23, wherein the mesenchymal stromal cells range from about 20 million cells to about 50 million cells, more preferably at about 25 million cells.

25. The method as claimed in claim 23, wherein the pooled allogeneic mesenchymal stromal cells are obtained by combining mesenchymal stromal cells from at least two donors, preferably about 2 donors to about 10 donors, more preferably about three donors.

26. The method as claimed in claim 23, wherein the pooled allogeneic mesenchymal stromal cells have plurality of HLA genotypes at loci selected from group comprising HLA-A, HLA-B, HLA-C, HLA-DQ and HLA-DR or any combinations thereof; and wherein the pooled mesenchymal stromal cells have sGAG (sulfated glycosaminoglycans) value ranging from about 25 μg to about 50 μg.

27. The method as claimed in claim 23, wherein at least 80% of the mesenchymal stromal cells are positive for cell specific markers selected from group comprising CD 73, CD 105, CD 44, CD90 and CD166 cells or any combinations thereof; and at least 90% of the mesenchymal stromal cells are negative for markers selected from group comprising CD34, CD45, CD133, CD14, CD19 and HLA-DR or any combinations thereof.

28. The method as claimed in claim 23, wherein the carrier is multiple electrolyte solution at a concentration ranging from about 80% to about 90%; the cyropreservant is Dimethyl Sulfoxide (DMSO) at concentration ranging from about 2% to about 10%, or cryopreservation medium comprising DMSO at concentration ranging from about 2% to about 10%; the protein is human serum albumin (HSA) at a concentration ranging from about 1% to about 6%.

29. The method as claimed in claim 23, wherein the composition is formulated as a formulation selected from group comprising aqueous suspension, emulsion, drop, emulsion in hard or soft gel capsule, elixir, lyophilized cell powder and cell spray or any combinations thereof, preferably aqueous suspension.

30. The method as claimed in claim 23, wherein the composition is administered as single dose or multiple doses, preferably as single dose, at one or multiple sites through mode selected from group comprising intraarticular administration, intramuscular administration, intravenous administration or any combinations thereof, preferably intraarticular administration.

31. The method as claimed in claim 23, wherein the subject is mammal selected from a group comprising human, horse, dog, camel, preferably human.

32. The method as claimed in claim 23, wherein the hyaluronic acid is administered to the subject prior to or post administering the composition comprising pooled mesenchymal stromal cells; preferably post administering the composition comprising pooled mesenchymal stromal cells.

33. The method as claimed in claim 23, wherein the hyaluronic acid is at an amount ranging from about 0.5 to about 6 ml, preferably about 2 ml; and the hyaluronic acid is administered at time duration ranging from about 30 seconds to about 24 hours, preferably about 30 seconds prior to or post administering the composition.

34. A kit for treating Osteoarthritis in subject in need thereof comprising composition as claimed in claim 16; and hyaluronic acid; optionally along with an instruction manual.

35. A method of assembling a kit as claimed in claim 34.
